(19) United States
(12) Patent Application Publication
(10) Pub. No.: US 2009/0098189 A1
(43) Pub. Date: Apr. 16, 2009

(54) AZAINDOLES AS INHIBITORS OF SOLUBLE ADENYLATE CYCLASE

(76) Inventors: Bernd BUCHMANN, Hohen Neuendorf (DE); Michael Haerter, Leverkusen (DE); Yolanda Cancho-Grande, Leverkusen (DE); Dirk Kosemund, Jena (DE); Hartmut Schirok, Wuppertal (DE); Duy Nguyen, Berlin (DE); Martin Fritsch, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/202,744

(22) Filed: Sep. 2, 2008

Related U.S. Application Data
(60) Provisional application No. 60/970,005, filed on Sep. 5, 2007.

(30) Foreign Application Priority Data
Sep. 5, 2007 (EP) 07075764.6

(51) Int. Cl. A61K 31/5377; C07D 471/04; A61K 31/437; A61P 15/16; A61P 15/18; A61K 9/02; C07D 413/14

(52) U.S. Cl. 424/433; 546/113; 514/300; 544/127; 514/233.8

(57) ABSTRACT

The present invention relates to azaindoles of general formula I, a method for the production thereof, and to the use thereof for the production of pharmaceutical compositions.

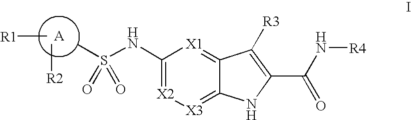

This application claims the benefit of the filing date of U.S. Provisional Application Ser. No. 60/970,005 filed Sep. 5, 2007.

The present invention relates to inhibitors of soluble adenylate cyclase, production thereof and use thereof for the production of a medicinal product for contraception.

A large number of modern methods of contraception may currently be used by women, but very few methods are available for male fertility control (condom and sterilization). Development of reliable new means of male fertility control is urgently needed. The infertility brought about by a “male pill” should be reversible and should be just as effective as the existing methods available to women. Infertility should be developed relatively quickly and should persist for as long as possible. Such a method of contraception should have no side-effects, and the preparations may be non-hormonal as well as hormonal. A possible starting point is regulation of the activity of an enzyme that plays an important role in fertilization of the ovum: soluble adenylate cyclase (sAC). This enzyme is mainly expressed in the testicular stem cells and is present in mature sperm.

In 1999, the authors Levin and Buck succeeded in purifying and cloning an isoform of sAC from rat testes (Proc. Natl. Acad. Sci. USA 96 (1): 79-84).

The recombinant rat enzyme can be stimulated by bicarbonate. It was demonstrated, using antibodies, that the catalytic domain of the enzyme is localized in testes, sperm, kidneys and the choroid plexus. These disclosures are the subject of application WO01/85753, which was granted in the USA (U.S. Pat. No. 6,544,768).

WO01/21829 (Conti et al.) claims isolated polynucleotide sequences that code for the human isoform of sAC, isolated sAC polypeptides and test systems, using which it is possible to identify substances that inhibit the activity of sAC. The possibility of using these substances to achieve a reversible reduction in the motile sperm count, and their use as a means of controlling male fertility, is disclosed.

John Herr's group demonstrated the isolation and characterization of the human isoform of sAC from sperm. WO 02/20745 also claims, in addition to nucleic acids that code for sAC, test systems for identifying substances that modulate the expression or the activity of human sAC. Such compounds might, for example, selectively inhibit the activity of sAC, as a consequence of which the sperm would lose the capacity to fertilize an ovum. These sAC inhibitors might therefore serve as medicinal products of non-hormonal contraception.

However, the sAC inhibitors that are already known display specific problems: catechol estrogens (T. Braun, Proc Soc Exp Biol Med 1990, 194(1): 58ff) and gossypol (K L Olgiati, Arch Biochem Biophys 1984, 231(2): 411ff) are inherently toxic, whereas adenosine analogs only have a very weak inhibitory action (M A Brown and E R Casillas, J Androl 1984, 5:361ff). The inhibitors of recombinant human sAC described by Zippin et al. are somewhat more potent (IC₅₀≦10 μM) (J H Zippin et al., J Cell Biol 2004, 164(4): 527ff).

The patent application WO 06/032541 (Bayer Schering Pharma) shows, for the first time, sAC inhibitors which are active within a range of 70 nM-10 μM. However, there continues to be a need to be able to provide compounds for male fertility control that are even more active.

It is an object of the present invention, therefore, to provide stable inhibitors of soluble adenylate cyclase.

This problem is solved by the provision of the compounds of general Formula I,

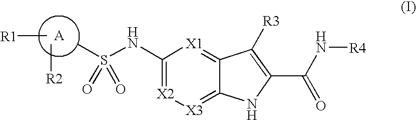

where:
A is a C₆-C₁₂-aryl or C₅-C₁₂-heteroaryl radical which can be optionally singly or multiply substituted with R¹ and/or R²,
R¹ is a hydrogen, halogen, amino,
   an —S(O)ₚ—C₁-C₆-alkyl group where p is 0-2,
   an SO₂NH₂, an —SO₂NH—C₁-C₆-alkyl group, an —NH—SO₂—C₁-C₆-alkyl group, a C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted,
      or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, amino, an —S(O)ₚ—C₁-C₆-alkyl group, where p is 0-2,
   an SO₂NH₂, —SO₂NH—C₁-C₆-alkyl group, —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂ or NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH(C₁-C₆-alkyl) or with CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted, or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cyclo-alkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ is a C₆-C₁₂-aryl,
   which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—, or
a C₅-C₁₆-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or
a C₃-C₆-cycloalkyl, which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
   a C₁-C₆-alkyl,
      which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl), CO—N(C₁-C₆-alkyl)₂ or C₆-C₁₂-aryl,
         which is optionally multiply substituted,
      or
      C₅-C₁₂-heteroaryl,
         which is optionally multiply substituted, or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
   and also their stereoisomers, diastereomers, enantiomers and salts, which overcome the known drawbacks and display improved properties, i.e. good efficacy, good solubility and stability.

The compounds according to the invention inhibit soluble adenylate cyclase and so prevent capacitation of the sperm and thus provide male fertility control.

The C₁-C₆- or the C₁-C₄-alkyl residue in R¹-R⁶ means in each case a linear or branched alkyl residue, such as methyl, ethyl, propyl, isopropyl, butyl, isobutyl, sec-butyl, tert-butyl, pentyl, isopentyl and hexyl. The alkyl residues can optionally also contain one or more double bonds; by way of example, mention may be made of ethenyl, 1-propenyl, 2-propenyl.

The alkyl residues can also optionally be substituted singly or multiply, identically or differently by halogen.

The C₁-C₆- or the C₁-C₄-alkoxy residue in R¹-R⁴ means in each case a linear or branched alkoxy residue, such as methoxy-, ethoxy-, n-propoxy-, iso-propoxy-, n-butoxy-, sec-butoxy-, iso-butoxy-, tert-butyloxy-, pentoxy-, iso-pentoxy- and hexoxy-.

The alkoxy residues can also be optionally substituted singly or multiply, identically or differently by halogen.

The C₁-C₆- or the C₁-C₄-acyl residue in R¹-R⁴ means in each case a linear or branched residue, such as formyl, acetyl, propionyl, butyroyl, iso-butyroyl, valeroyl and benzoyl.

The acyl residues can optionally be additionally substituted singly or multiply, identically or differently by halogen.

A C₁-C₆- or the C₁-C₄-alkylene residue in R³ means in each case a linear or branched residue, the reference being, for example, to the following residues: methylene, ethylene, propylene, butylene, pentylene, hexylene.

C₃-C₆-Cycloalkyl in R¹-R⁴ means monocyclic alkyl rings such as cyclopropyl, cyclobutyl, cyclopentyl and cyclohexyl.

The cycloalkyl residues can also contain an SO₂ group or one or two keto groups or one or more heteroatoms, such as oxygen, sulfur and/or nitrogen, instead of the carbon atoms. Such heterocycloalkyls with 3 to 6 ring atoms are preferred, such as, for example, aziridinyl, oxetanyl, tetrahydrofuranyl, pyrrolidinyl, morpholinyl, thiomorpholinyl, piperidinyl, tetrahydropyranyl, dioxanyl and piperazinyl. The ring systems, in which optionally one or more possible double bonds can be contained in the ring, mean for example cycloalkenyls such as cyclopropenyl, cyclobutenyl, cyclopentenyl, cyclopentadienyl, cyclohexenyl, cycloheptenyl, where the coupling can take place both on the double bond and on the single bonds.

Halogen means in each case fluorine, chlorine, bromine or iodine.

In each case the aryl residue in R¹-R⁴ comprises 6-12 carbon atoms and can for example be benzocondensed. The following may be mentioned as examples: phenyl, tropyl, cyclooctadienyl, indenyl, naphthyl, biphenyl, florenyl, anthracenyl, etc.

In each case the heteroaryl residue in R¹-R⁴ comprises 5 to at most 16 ring atoms and can contain one or more, identical or different heteroatoms, such as oxygen, sulfur or nitrogen in the ring instead of carbon, and can be mono-, bi- or tricyclic and can additionally be benzocondensed in each case.

The following may be mentioned as examples:

thienyl, furanyl, pyrrolyl, oxazolyl, imidazolyl, pyrazolyl, isoxazolyl, isothiazolyl, oxadiazolyl, triazolyl, thiadiazolyl, etc. and benzo derivatives thereof, e.g. benzofuranyl, benzothienyl, benzooxazolyl, benzimidazolyl, indazolyl, indolyl, isoindolyl, etc; or pyridazinyl, pyrimidinyl, pyrazinyl, triazinyl, etc. and benzo derivatives thereof, e.g. quinolyl, isoquinolyl, etc; or azocinyl, indolizinyl, purinyl, etc. and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, etc.

The heteroaryl residue can be benzocondensed in each case. For example, the following may be mentioned as 5-ring heteroaromatics: thiophene, furan, oxazole, thiazole, imidazole, pyrazole and benzo derivatives thereof and as 6-ring-heteroaromatics pyridine, pyrimidine, triazine, quinoline, isoquinoline and benzo derivatives.

Heteroatoms is to be taken to mean oxygen, nitrogen or sulfur atoms.

If an acid function is present, the physiologically compatible salts of organic and inorganic bases are suitable as salts, such as the readily soluble alkali and alkaline-earth salts and N-methyl-glucamine, dimethyl-glucamine, ethyl-glucamine, lysine, 1,6-hexadiamine, ethanolamine, glucosamine, sarcosine, serinol, tris-hydroxymethylaminomethane, aminopropanediol, Sovak base, 1-amino-2,3,4-butanetriol.

If a basic function is present, the physiologically compatible salts of organic and inorganic acids are suitable, such as hydrochloric acid, sulfuric acid, phosphoric acid, citric acid, tartaric acid, oxalic acid, etc.

Particular preference is given to those compounds of general formula (I) where
A is a phenyl, naphthyl or C₅-C₁₂-heteroaryl radical which can be optionally singly or multiply substituted with R¹ and/or R²,
R¹ is a hydrogen, halogen, amino,
   an —S(O)ₚ—C₁-C₆-alkyl group, where p is 0-2,
   an SO₂NH₂, —SO₂NH—C₁-C₆-alkyl group, —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted,
      or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, amino, an —S(O)ₚ—C₁-C₆-alkyl group, where p is 0-2,
   an SO₂NH₂, an —SO₂NH—C₁-C₆-alkyl group, an —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂ or an NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH(C₁-C₆-alkyl) or with CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted,
      or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ is a C₆-C₁₂-aryl,
   which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—,
or
a C₅-C₁₆-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or
a C₃-C₆-cycloalkyl,
   which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
   a C₁-C₆-alkyl,
      which can be optionally singly or multiply, identically or differently substituted with an —S(O)ₚ—C₁-C₆-alkyl group or an —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy,
   or
   a C₆-C₁₂-aryl,
   which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl or a monocyclic C₅-C₇-heteroaryl radical which can be optionally singly or multiply substituted with R¹ and/or R²,
R¹ is a hydrogen, halogen, amino, an —S(O)ₚ—C₁-C₆-alkyl group, where p is 0-2,
   an SO₂NH₂, an —SO₂NH—C₁-C₆-alkyl group, an —NH—SO₂—C₁-C₆-alkyl group, C₁-C₆-acyl, NH—CO—NH₂, —O—CO—NH(C₁-C₆-alkyl), —O—CO—N(C₁-C₆— alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or a C₁-C₆-alkyl,
      which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted,
   or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2,
   or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, amino, an —S(O)ₚ—C₁-C₆-alkyl group, where p is 0-2,
   an SO₂NH₂, an —SO₂NH—C₁-C₆-alkyl group, an —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂ or NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH(C₁-C₆-alkyl) or with CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted,
   or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ is a C₆-C₁₂-aryl,
   which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₂-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—, or
a C₅-C₁₆-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂, or
a C₃-C₆-cycloalkyl,
   which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
   a C₁-C₆-alkyl,
      which can be optionally singly or multiply, identically or differently substituted with an —S(O)ₚ—C₁-C₆-alkyl group or an —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
         which is optionally multiply substituted,
      C₅-C₁₂-heteroaryl,
         which is optionally multiply substituted, or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl or a monocyclic C₅-C₇-heteroaryl radical which can be optionally singly or multiply substituted with R¹ and/or R²,
R¹ is a hydrogen, halogen, amino, an —S(O)ₚ—C₁-C₆-alkyl group, where p is 0-2,
   an SO₂NH₂, an —SO₂NH—C₁-C₆-alkyl group, an —NH—SO₂—C₁-C₆-alkyl group, C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
      which is optionally multiply substituted,
   C₆-C₁₂-aryl,
      which is optionally multiply substituted,
   or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂, or
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₆-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, C₁-C₆-alkyl, C₁-C₆-alkoxy, C₁-C₆-acyl-, C₆-C₁₂-aryl, C₅-C₁₆-heteroaryl, C₃-C₆-cycloalkyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, O—C₆-C₁₂-aryl, O—C₅-C₁₂-heteroaryl, or
R³ is a C₆-C₁₂-aryl,
   which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—, or
a C₅-C₁₆-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or
a C₃-C₆-cycloalkyl,
   which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
   a C₁-C₆-alkyl,
      which can be optionally singly or multiply, identically or differently substituted with an —S(O)ₚ—C₁-C₆-alkyl group or an —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl or a monocyclic C₅-C₇-heteroaryl radical which can be optionally singly or multiply substituted with R¹ and/or R²,
R¹ is a hydrogen, halogen, amino, an —S(O)ₚ—C₁-C₆-alkyl group, where p is 0-2,
   an SO₂NH₂, an —SO₂NH—C₁-C₆-alkyl group, an —NH—SO₂—C₁-C₆-alkyl group, C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
         which is optionally multiply substituted,
      C₆-C₁₂-aryl,
         which is optionally multiply substituted,
   or
   a C₁-C₆-alkoxy,
      which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂, or
   an O—C₆-C₁₂-aryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   an O—C₅-C₁₂-heteroaryl,
      which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
   a C₆-C₁₂-aryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a C₅-C₁₂-heteroaryl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, C₁-C₆-acyl-, NH—CO—NH₂, —O—CO—NH(C₁-C₆-alkyl), —O—CO—N(C₁-C₆-alkyl)₂, an NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, C₁-C₆-alkyl, C₁-C₆-alkoxy, O—C₆-C₁₂-aryl, O—C₅-C₁₂-heteroaryl, C₆-C₁₂-aryl, C₅-C₁₆-heteroaryl, C₃-C₆-cycloalkyl,
R³ is a C₆-C₁₂-aryl,
   which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—, or
a C₅-C₁₂-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
   a C₁-C₆-alkyl,
      which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cyclo-alkyl), CO—NH(C₁-C₆-alkyl) or with CO—N(C₁-C₆-alkyl)₂, or C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl or a monocyclic C₅-C₇-heteroaryl radical which can be optionally singly or multiply substituted with R¹ and/or R²,
R¹ is a hydrogen, halogen, an —S(O)ₚ—C₁-C₆-alkyl group, where p is 0-2, an SO₂NH₂, an —SO₂NH—C₁-C₆-alkyl group, an —NH—SO₂—C₁-C₆-alkyl group, or
   a C₁-C₆-acyl, hydroxy, cyano, CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
   a C₁-C₆-alkyl,
      which can be optionally singly or multiply, identically or differently substituted with C₁-C₆-alkoxy, hydroxy, cyano, N—(C₁-C₆-alkyl)₂, C₅-C₁₂-heteroaryl,
         which is optionally singly or multiply, identically or differently substituted with halogen or C₁-C₆-alkyl,
   C₆-C₁₂-aryl,
      which is optionally singly or multiply, identically or differently substituted with halogen or C₁-C₆-alkyl, or
   a C₁-C₆-alkoxy, or
   an O—C₆-C₁₂-aryl,
      which can be optionally singly or multiply, identically or differently substituted with halogen or C₁-C₆-alkyl, C₃-C₆-cycloalkyl or C₁-C₆-alkoxy, or
   an O—C₅-C₁₂-heteroaryl,
      which can be optionally singly or multiply, identically or differently substituted with halogen or C₁-C₆-alkyl, C₃-C₆-cycloalkyl or C₁-C₆-alkoxy, or
   a phenyl,
      which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a monocyclic C₅-C₇-heteroaryl radical,
      which can be optionally singly or multiply, identically or differently substituted with halogen, with C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, with C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen,
R³ is a C₆-C₁₂-aryl,
   which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—, or
a C₅-C₁₂-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
   a C₁-C₆-alkyl,
      which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl, thienyl or pyridinyl radical which can be optionally singly or doubly substituted with R¹ and/or R²,
R¹ is a hydrogen, halogen, C₁-C₆-alkoxy
   a C₁-C₆-alkyl,
      which can be optionally singly or multiply, identically or differently substituted with C₁-C₆-alkoxy, hydroxy, cyano, N—(C₁-C₆-alkyl)₂, phenyl,
   an O—C₆-C₁₂-aryl or an O—C₅-C₁₂-heteroaryl, or
   a phenyl,
      which can be optionally singly or doubly, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a thienyl or pyridinyl radical,
      which can be optionally singly or doubly, identically or differently substituted with halogen, with C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, with C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen,
R³ is a C₆-C₁₂-aryl,
   which can be optionally singly or doubly, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
      which is optionally singly or multiply substituted,
   or
   with two adjacent positions can be substituted by —O—CH₂—O—, —O—CH₂—CH₂—O—, or —O—C(CH₃)₂—O—, or
a C₅-C₁₂-heteroaryl,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
   a C₁-C₆-alkyl,
      which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl, thienyl or pyridinyl radical which can be optionally singly or doubly substituted with R¹ and/or R²,
R¹ is a hydrogen, halogen, C₁-C₆-alkoxy
   a C₁-C₆-alkyl,
      which can be optionally singly or multiply, identically or differently substituted with C₁-C₆-alkoxy, hydroxy, cyano, N—(C₁-C₆-alkyl)₂, phenyl,
   an O—C₆-C₁₂-aryl or an O—C₅-C₁₂-heteroaryl, or
   a phenyl,
      which can be optionally singly or doubly, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a thienyl or pyridinyl radical,
      which can be optionally singly or doubly, identically or differently substituted with halogen, with C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, with C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen,
R³ is a phenyl,
   which can be optionally singly or doubly, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl, which is optionally singly or multiply substituted,
   or
   with two adjacent positions can be substituted by —O—CH₂—O—, —O—CH₂—CH₂—O—, or —O—C(CH₃)₂—O—, or
a thiophenyl, furanyl or pyridinyl radical,
   which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
   a C₁-C₆-alkyl,
      which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
   a C₆-C₁₂-aryl,
      which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
   a C₅-C₁₂-heteroaryl,
      which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X² is a nitrogen if X¹ and X³ are a CH group, or
X³ is a nitrogen if X¹ and X² are a CH group,
   and also their stereoisomers, diastereomers, enantiomers and salts.

Preference is likewise given to those compounds of general formula I where
A is a phenyl, thienyl or pyridinyl radical which can be optionally singly or doubly substituted with R¹ and/or R²,
R¹ is a hydrogen, halogen, C₁-C₄-alkoxy
   a C₁-C₄-alkyl,
      which can be optionally singly or multiply, identically or differently substituted with C₁-C₄-alkoxy, hydroxy, cyano, N—(C₁-C₄-alkyl)₂, phenyl,
   an O—C₆-C₁₂-aryl or an O—C₅-C₁₂-heteroaryl, or
   a phenyl,
      which can be optionally singly or doubly, identically or differently substituted with halogen, C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxy, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH—(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl) or an —S(O)ᵣ—CH₃, where r is 0-2, or
   two adjacent positions can be substituted by —O—CH₂—O— or —O—C(CH₃)₂—O—, or
   a thienyl or pyridinyl radical,
      which can be optionally singly or doubly, identically or differently substituted with halogen, with C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxy, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl) or an —S(O)ᵣ—CH₃, where r is 0-2, or
   a C₃-C₆-cycloalkyl,
      which can be optionally singly or multiply, identically or differently substituted with halogen, with C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R² is a hydrogen,
R³ is a phenyl,
   which can be optionally singly or doubly, identically or differently substituted with halogen, C₁-C₄-alkyl, C₁-C₄-alkoxy, hydroxy, C₁-C₄-alkylene-OH, C₁-C₄-alkylene-NR⁵R⁶, cyano, CO₂—(C₁-C₄-alkyl), N—(C₁-C₄-alkyl)₂, CO—NH—(C₁-C₄-alkyl), CO—NH—(C₁-C₄-alkylene-OH), CO—NH—(C₁-C₄-alkylene-NR⁵R⁶), CO—N(C₁-C₄-alkyl)₂ or C₁-C₄-acyl,
      which is optionally singly or multiply substituted,
   or
   with two adjacent positions can be substituted by —O—CH₂—O—, —O—CH₂—CH₂—O—, or —O—C(CH₃)₂—O—, or
a thiophenyl, furanyl or pyridinyl radical,
   which can be optionally singly or multiply, identically or differently substituted with halogen, C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH—(C₁-C₄-alkylene-OH), CO—NH—(C₁-C₄-alkylene-NR⁵R⁶), CO—NH(C₁-C₄-alkyl) or CO—N(C₁-C₄-alkyl)₂,
R⁴ is a hydrogen,
   a C₁-C₄-alkyl,
      which can be optionally singly or multiply, identically or differently substituted with an —S(O)ₚ—C₁-C₄-alkyl group or —S(O)ₚ—C₃-C₆-cycloalkyl group, where p is 0-2, hydroxy, cyano, COOH, CO—NH₂, SO₂NH₂, SO₂NH—(C₁-C₄-alkyl), C₃-C₆-cycloalkyl, C₁-C₄-acyl, C₁-C₄-alkoxy, CO₂—(C₁-C₄-alkyl), N—(C₁-C₄-alkyl)₂, NH—(C₁-C₄-alkyl), NH—(C₃-C₆-cycloalkyl), CO—NH(C₁-C₄-alkyl) or with CO—N(C₁-C₄-alkyl)₂, C₆-C₁₂-aryl,
         which is optionally multiply substituted,
      C₅-C₁₂-heteroaryl,
         which is optionally multiply substituted,
   or
   a C₃-C₆-cycloalkyl,
      which can be optionally singly or multiply, identically or differently substituted with hydroxy, CH₂—OH, cyano, COOH, CO—NH₂, SO₂NH₂, C₁-C₄-alkyl, C₁-C₄-acyl or C₁-C₄-alkoxy, or
   a C₆-C₁₂-aryl,
      which can be optionally singly or multiply, identically or differently substituted with halogen, hydroxy, C₁-C₄-alkyl, C₁-C₄-acyl, C₁-C₄-alkoxy, N—(C₁-C₄-alkyl)₂, NH—(C₁-C₄-alkyl) or cyano, or
   a C₅-C₁₂-heteroaryl,
      which can be optionally singly or multiply, identically or differently substituted with halogen, amino, hydroxy, cyano, COOH, C₁-C₄-alkyl, C₁-C₄-acyl, C₁-C₄-alkoxy, N—(C₁-C₄-alkyl)₂ or NH—(C₁-C₄-alkyl),
R⁵, R⁶ are independently of one another a hydrogen or a C₁-C₄-alkyl, and
X² is a nitrogen if X¹ and X³ are a CH group, or
X³ is a nitrogen if X¹ and X² are a CH group,
   and also their stereoisomers, diastereomers, enantiomers and salts.

The following compounds according to the present invention are quite especially preferred:
1. N-(Tetrahydropyran-4-yl)-5-(4-tert-butylphenylsulfonylamino)-3-phenyl-1H-pyrrolo[3,2-b]pyridine-2-carboxamide
2. N-(2-Morpholin-4-ylethyl)-5-(4-tert-butylphenylsulfonylamino)-3-phenyl-1H-pyrrolo[3,2-b]pyridine-2-carboxamide
3. N-(2-Morpholin-4-ylethyl)-5-(4-tert-butylphenylsulfonylamino)-3-phenyl-1H-pyrrolo[2,3-c]pyridine-2-carboxamide
4. N-(2-Morpholin-4-ylethyl)-5-(4-tert-butyl-phenylsulfonylamino)-3-phenyl-1H-pyrrolo[2,3-b]pyridine-2-carboxamide
5. (±)-N-(2-Hydroxypropyl)-5-(4-tert-butylphenylsulfonylamino)-3-phenyl-1H-pyrrolo[2,3-b]pyridine-2-carboxamide
6. N-(Tetrahydropyran-4-yl)-5-(4-tert-butylphenylsulfonylamino)-3-phenyl-1H-pyrrolo[2,3-b]pyridine-2-carboxamide
7. (±)-N-(2-Hydroxy-1-methylethyl)-5-(4-tert-butylphenylsulfonylamino)-3-phenyl-1H-pyrrolo[2,3-b]pyridine-2-carboxamide
8. (1S,2R)—N-(2-Hydroxycyclopentyl)-5-(4-tert-butylphenylsulfonylamino)-3-phenyl-1H-pyrrolo[2,3-b]pyridine-2-carboxamide
9. N-(2-Morpholin-4-ylethyl)-3-phenyl-5-(4-trifluoromethylphenylsulfonylamino)-1H-pyrrolo[2,3-b]pyridine-2-carboxamide
10. (1S,2R)—N-(2-Hydroxycyclopentyl)-5-(biphenyl-4-sulfonylamino)-3-phenyl-1H-pyrrolo[2,3-b]pyridine-2-carboxamide
11. N-(2-Morpholin-4-ylethyl)-3-phenyl-5-(4-trifluoromethoxyphenylsulfonyl-amino)-1H-pyrrolo[2,3-b]pyridine-2-carboxamide
12. N-(2-Morpholin-4-ylethyl)-5-(biphenyl-4-sulfonylamino)-3-phenyl-1H-pyrrolo[2,3-b]pyridine-2-carboxamide
13. (1S,2R)—N-(2-Hydroxycyclopentyl)-3-phenyl-5-(4-trifluoromethylphenyl-sulfonylamino)-1H-pyrrolo[2,3-b]pyridine-2-carboxamide
14. N-[2-(Morpholine-4-sulfonyl)ethyl]-5-(4-tert-butylphenylsulfonylamino)-3-phenyl-1H-pyrrolo[2,3-b]pyridine-2-carboxamide
15. N-(Pyridin-4-yl)-5-(4-tert-butylphenylsulfonylamino)-3-phenyl-1H-pyrrolo[2,3-b]pyridine-2-carboxamide
16. N-(Thiazol-2-yl)-5-(4-tert-butylphenylsulfonylamino)-3-phenyl-1H-pyrrolo-[2,3-b]pyridine-2-carboxamide
17. N-(Pyridin-3-ylmethyl)-5-(4-tert-butyl-phenylsulfonylamino)-3-phenyl-1H-pyrrolo[2,3-b]pyridine-2-carboxamide
18. (±)-N-(Tetrahydrofuran-2-ylmethyl)-5-(4-tert-butylphenylsulfonylamino)-3-phenyl-1H-pyrrolo[2,3-b]pyridine-2-carboxamide
19. N-(Ethyl)-5-(4-tert-butylphenylsulfonylamino)-3-phenyl-1H-pyrrolo[2,3-b]-pyridine-2-carboxamide
20. N-[2-(2-Oxoimidazolidin-1-yl)ethyl]-5-(4-tert-butylphenylsulfonylamino)-3-phenyl-1H-pyrrolo[2,3-b]pyridine-2-carboxamide
21. (1S,2R)—N-(2-Hydroxycyclopentyl)-3-phenyl-5-(4-trifluoromethoxyphenyl-sulfonylamino)-1H-pyrrolo[2,3-b]pyridine-2-carboxamide
22. 5-(4-tert-Butylphenylsulfonylamino)-3-phenyl-1H-pyrrolo[2,3-b]pyridine-2-carboxamide
23. N-(2-Sulfamoylethyl)-5-(4-tert-butylphenylsulfonylamino)-3-phenyl-1H-pyrrolo[2,3-b]pyridine-2-carboxamide
24. 5-(Biphenyl-4-sulfonylamino)-3-phenyl-1H-pyrrolo[2,3-b]pyridine-2-carboxamide
25. (1S,2R)—N-(2-Hydroxycyclopentyl)-3-benzo[1,3]dioxol-5-yl-5-(4-trifluoro-methoxyphenylsulfonylamino)-1H-pyrrolo[2,3-b]pyridine-2-carboxamide
26. (1S,2R)—N-(2-Hydroxycyclopentyl)-3-thiophen-2-yl-5-(4-trifluoromethoxy-phenylsulfonylamino)-1H-pyrrolo[2,3-b]pyridine-2-carboxamide
27. (1S,2R)—N-(2-Hydroxycyclopentyl)-3-thiophen-3-yl-5-(4-trifluoromethoxy-phenylsulfonylamino)-1H-pyrrolo[2,3-b]pyridine-2-carboxamide
28. (1S,2R)—N-(2-Hydroxycyclopentyl)-3-furan-2-yl-5-(4-trifluoromethoxy-phenylsulfonylamino)-1H-pyrrolo[2,3-b]pyridine-2-carboxamide
29. (1S,2R)—N-(2-Hydroxycyclopentyl)-3-furan-3-yl-5-(4-trifluoromethoxy-phenylsulfonylamino)-1H-pyrrolo[2,3-b]pyridine-2-carboxamide
30. (1S,2R)—N-(2-Hydroxycyclopentyl)-3-benzo[1,3]dioxol-5-yl-5-(4-tert-butyl-phenylsulfonylamino)-1H-pyrrolo[2,3-b]pyridine-2-carboxamide
31. (1S,2R)—N-(2-Hydroxycyclopentyl)-3-thiophen-2-yl-5-(4-tert-butylphenyl-sulfonylamino)-1H-pyrrolo[2,3-b]pyridine-2-carboxamide
32. (1S,2R)—N-(2-Hydroxycyclopentyl)-3-thiophen-3-yl-5-(4-tert-butylphenyl-sulfonylamino)-1H-pyrrolo[2,3-b]pyridine-2-carboxamide
33. (1S,2R)—N-(2-Hydroxycyclopentyl)-3-furan-2-yl-5-(4-tert-butylphenyl-sulfonylamino)-1H-pyrrolo[2,3-b]pyridine-2-carboxamide
34. (1S,2R)—N-(2-Hydroxycyclopentyl)-3-furan-3-yl-5-(4-tert-butylphenyl-sulfonylamino)-1H-pyrrolo[2,3-b]pyridine-2-carboxamide
35. (±)-N-(Tetrahydrofuran-2-ylmethyl)-3-thiophen-3-yl-5-(4-trifluoromethoxy-phenylsulfonylamino)-1H-pyrrolo[2,3-b]pyridine-2-carboxamide
36. (±)-N-(Tetrahydrofuran-2-ylmethyl)-3-thiophen-2-yl-5-(4-trifluoromethoxy-phenylsulfonylamino)-1H-pyrrolo[2,3-b]pyridine-2-carboxamide
37. (±)-N-(Tetrahydrofuran-2-ylmethyl)-3-thiophen-3-yl-5-(4-tert-butylphenyl-sulfonylamino)-1H-pyrrolo[2,3-b]pyridine-2-carboxamide
38. (±)-N-(Tetrahydrofuran-2-ylmethyl)-3-thiophen-2-yl-5-(4-tert-butyl-phenylsulfonylamino)-1H-pyrrolo[2,3-b]pyridine-2-carboxamide

The invention additionally relates to a process for the preparation of the compounds of general formula I according to the invention, which comprises reacting a compound of formula II

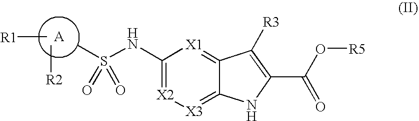

in which R¹, R², R³, X¹, X² and X³ have the definitions given above and R⁵ can be a hydrogen or a C₁-C₆-alkyl radical, preference being given to hydrogen or to the methyl or ethyl radical, with an amine of general formula III

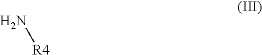

in which R⁴ has the definition given above, by methods that are known to the skilled person, and/or any required protecting groups are subsequently cleaved and/or any double bonds present are hydrogenated.

Where R⁵ is hydrogen the reaction may take place first of all by activation of the acid function, in which case, for example, the carboxylic acid of general formula II is first converted into the mixed anhydride in the presence of a tertiary amine, such as triethylamine, for example, with isobutyl chloroformate. The reaction of the mixed anhydride with the alkali metal salt of the corresponding amine takes place in an inert solvent or solvent mixture, such as tetrahydrofuran, dimethoxyethane, dimethylformamide or hexamethyl-phosphoramide, for example, at temperatures between −30° C. and +60° C., preferably at 0° C. to 30° C.

A further possibility is to activate the carboxylic acid of general formula II by means of reagents, such as HOBt or HATU, for example. The acid is reacted, for example, with HATU in an inert solvent, such as DMF, for example, in the presence of the corresponding amine of general formula III and with a tertiary amine, such as ethyldiisopropylamine, for example, at temperatures between −50 and +60° C., preferably at 0° C. to 30° C.

Where R⁵ is C₁-C₆-alkyl it is also possible, for example, to carry out direct amidolysis of the ester with the corresponding amine, optionally with the assistance of aluminum trialkyl reagents, preferably aluminum trimethyl.

### Synthesis of the 4- and 6-Azaindoles:

Where X¹ is a nitrogen and X² and X³ are a CH group, or X² is a nitrogen and X¹ and X³ are a CH group:

the compounds of general formula II which serve as starting materials can be prepared, for example, by conventionally reacting the amino group in the aza-indole esters of general formula IV

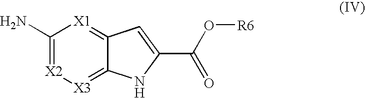

whose preparation is described in the experimental section, and
in which R⁶ is a C₁-C₆-alkyl radical, preferably a methyl or ethyl radical, with a sulfonic acid derivative of general formula V

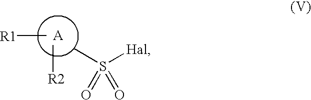

in which R¹, R² and A have the definitions given above and Hal is a halogen, preferably chloride or bromide, in the presence of a base such as pyridine, diisopropylethylamine, triethylamine or potassium carbonate, for example, to give the compounds of general formula VI

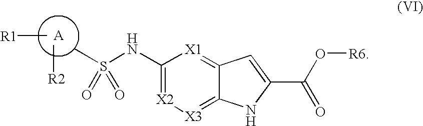

The esters of general formula VI are then halogenated in position 3, for example, by means of iodine, NBI, NBS or else CuBr₂, then provided on an intermediate basis with a nitrogen-protecting group, as for example by means of stirring in Boc₂O with the Boc protecting group, and subsequently converted in a Pd-catalyzed reaction with boronic acid derivatives of general formula VII

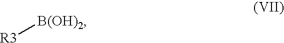

in which R³ has the definition given above, and subsequent elimination of the protecting groups, in the case of the Boc protecting group by means of acids such as trifluoroacetic acid, for example, and where necessary by hydrolysis and subsequent esterification with R⁵—OH, into the esters of general formula II

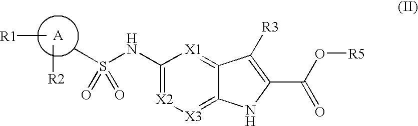

in which R¹, R², R³, R⁵ and A have the definitions given above and X¹ is a nitrogen and X² and X³ are a CH group, or X² is a nitrogen and X¹ and X³ are a CH group.

### Synthesis of the 7-Azaindoles:

Where X³ is a nitrogen and X¹ and X² are a CH group:

The compounds of general formula II that serve as starting materials, with the given definition of X¹, X² and X³, can be prepared, for example, by conventionally reacting the known acid chloride VIII

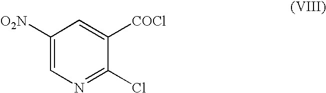

initially under Friedel-Crafts conditions with the aryl or heteroaryl derivatives of general formula IX

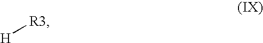

in which R³ has the definition given above, in the presence of a catalyst, such as AlCl₃ or Ln(OTf)₃, for example, to give the compounds of general formula X

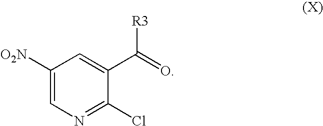

An alternative possibility for arriving at the compounds of general formula X would also be to convert the aryl or heteroaryl derivatives of general formula XI

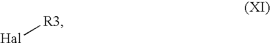

in which R³ has the definition given above and Hal is a halogen atom, preferably an iodine, bromine or chlorine, into the corresponding Grignard compound, using magnesium, or into the corresponding organolithium compound, using lithium or t-butyllithium, and then, by methods known to the skilled person, to convert these compounds into the ketones of general formula X, with the acid chloride of general formula VIII, where appropriate with addition of cadmium dichloride.

The ketones of general formula X are then reacted in position 2, by means of nucleophilic substitution, for example, with the known p-methoxybenzyl-protected glycine ester XII

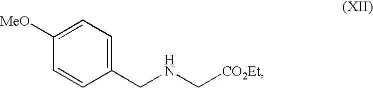

and the resulting product is reacted by means of acetic anhydride in pyridine or else by bases, such as LDA, LiN(TMS)₂, potassium carbonate or diisopropylethylamine, for example, in an inert solvent at temperatures between 0° C. and the boiling temperature of the solvent, such as tetrahydrofuran, for example, to give the compounds of general formula XIII

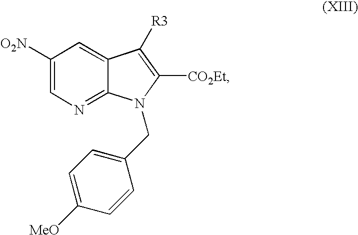

in which R³ has the definition given above.

The p-methoxybenzyl protecting group in the derivatives XIII can be eliminated, for example, by trifluoroacetic acid or else AlCl₃ in anisole. The nitro compound obtained is then converted into the amines of general formula XIV by stirring in a hydrogen atmosphere, where appropriate under pressure, in the presence of a catalyst such as palladium on carbon, for example. For the purification of these compounds it may if appropriate also be necessary to convert the free bases into salts, as for example into the hydrochloride by stirring with HCl in dioxane;

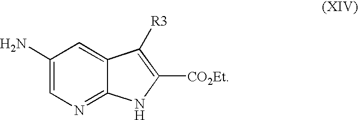

The resulting amines or their salts are then converted by reaction with a sulfonic acid derivative of general formula V

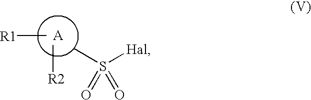

in which R¹, R² and A have the definitions given above and Hal is a halogen, preferably chloride or bromide, in the presence of a base, such as pyridine, diisopropylethylamine, triethylamine or potassium carbonate, for example, and, where appropriate, after hydrolysis of the ethyl ester and/or by subsequent esterification with R⁵—OH, by methods that are known to the skilled person, into the esters of general formula II

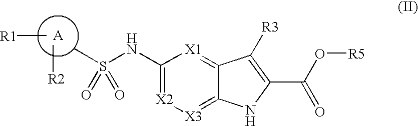

in which R¹, R², R³, R⁵ and A have the definitions given above and X¹ and X² are a CH group and X³ is a nitrogen.

The compounds according to the invention inhibit soluble adenylate cyclase, and this is also the basis of their action for example in male fertility control.

Adenylate cyclases are the effector molecules for one of the most-used signal-transduction pathways; they synthesize the second messenger molecule cyclic adenosine monophosphate (cAMP) from adenosine triphosphate (ATP) with elimination of pyrophosphate (PP). cAMP mediates numerous cellular responses to a large number of neurotransmitters and hormones.

Soluble, sperm-specific adenylate cyclase (sAC, human mRNA sequence (GenBank) nm_{—}018417, human gene ADCY X) is one out of ten adenylate cyclases described in the human genome. sAC exhibits some specific properties that distinguish it from the other adenylate cyclases. In contrast to all other adenylate cyclases, sAC is stimulated by the concentration of bicarbonate in the surrounding medium and not by G proteins. sAC does not possess any transmembrane regions in its amino acid sequence, it cannot be inhibited by forskolin, can be stimulated much more strongly by manganese than by magnesium, and only displays slight sequence homologies to the other adenylate cyclases (≦26% identity of the catalytic domains I and II of sAC with other adenylate cyclases at the amino acid level).

Specific, manganese-dependent activity of sAC was first described by T. Braun et al. (1975, PNAS 73:1097ff) in rat testis and sperm. N. Okamura et al. (1985, J. Biol. Chem. 260(17):9699ff) showed that the substance which stimulates the activity of sAC in boar seminal fluid is bicarbonate. It was also shown that AC activity that can be stimulated by bicarbonate can only be detected in rat testis and sperm, but not in other tissues. sAC was purified from rat testis and sequenced for the first time by the Buck and Levin group (J. Buck et al. 1999, PNAS 96:79ff, WO 01/85753). The expected properties (e.g. capacity to be stimulated by bicarbonate and magnesium) were confirmed on recombinantly expressed protein (Y. Chen et al. 2000, Science 289:625ff).

Testis-specific and sperm-specific expression of the enzymes can be concluded from data on the distribution of sAC mRNA and on sAC activity that can be stimulated by bicarbonate (M L Sinclair et al. 2000, Mol Reprod Develop 56:6ff; N Okamura et al. 1985, J. Biol. Chem. 260(17):9699ff; J. Buck et al. 1999, PNAS 96:79ff). In the testis, sAC mRNA is only expressed in later stages of the gametes developing to sperm, but not in somatic cells (M L Sinclair et al. 2000, Mol Reprod Develop 56:6ff).

There have been a number of pharmacological investigations into the function of sAC in sperm in mammals. Before sperm can penetrate the zona pellucida of the ovum and then fuse with the oolemma of the ovum, sperm must be prepared for this functionality. This process, sperm capacitation, has been thoroughly investigated. A capacitated sperm is characterized by an altered pattern of movement and by the ability to go through the process of the acrosome reaction (release of lytic enzymes which presumably serve for penetration of the zona pellucida by the sperm) when suitably stimulated. Sperm capacitation takes place in vivo and in vitro and among other things independently of a raised bicarbonate concentration in the medium (P E Visconti & G S Kopf (1998), Biol Reprod 59:1ff; E de Lamirande et al. 1997, Mol Hum Reprod 3(3):175ff). Sperm capacitation can also be stimulated by adding suitable membrane-passing cAMP analogs, e.g. db-cAMP, and an inhibitor that prevents their degradation (e.g. IBMX). The presumed dependence of sperm function on sAC was confirmed only recently by a genetic deletion model, a so-called knock-out mouse (G Esposito et al. 2004, PNAS 101 (9):2993ff). Male mice lacking the gene for sAC exhibit spermatogenesis that proceeds normally, but are infertile. The sperm have motility defects and are not capable of fertilizing an egg. The animals did not display any other defects or abnormal findings, which contradicts other hypothesized functions of sAC (J H Zippin et al. 2003, FASEB 17:82ff)).

sAC has a unique sequence and only slight homology to other somatic adenylate cyclases. It is the only adenylate cyclase in mammalian sperm and the activity is essential for sperm motility and capacitation. Specific sAC inhibitors accordingly represent an important possibility for controlling male fertility.

The compounds according to the invention of general formula I are excellent inhibitors of soluble adenylate cyclase.

The present invention relates to medicinal products that contain at least one of the compounds of formula I.

The present invention also relates to medicinal products that comprise the compounds according to the invention with suitable vehicles and excipients.

For use of the compounds according to the invention as medicinal products they are converted to the form of a pharmaceutical preparation, which contains, in addition to the active substance, pharmaceutical, organic or inorganic inert vehicles that are suitable for enteral or parenteral application, for example water, gelatin, gum arabic, lactose, starch, magnesium stearate, talc, vegetable oils, polyalkylene glycols etc. The pharmaceutical preparations can be in solid form, for example as tablets, film-coated tablets, suppositories, or capsules, or in liquid form, for example as solutions, suspensions or emulsions. If necessary they also contain excipients, such as preservatives, stabilizers, wetting agents or emulsifiers; salts for altering osmotic pressure or buffers. These pharmaceutical preparations are also the object of the present invention.

Injection solutions or suspensions, especially aqueous solutions of the active compounds in polyhydroxyethoxylated castor oil, are particularly suitable for parenteral application.

Surface-active excipients such as salts of bile acids or animal or vegetable phospholipids, as well as mixtures thereof and liposomes or their constituents, can also be used as carrier systems.

In particular, tablets, film-coated tablets or capsules with talc and/or hydrocarbon vehicles or binders, for example lactose, maize starch or potato starch, are suitable for oral application. Application can also be in liquid form, for example as juice, to which a sweetener is added if required.

Suppositories, for example, are suitable and customary for vaginal application.

The present invention also relates to enteral, parenteral, vaginal and oral application.

The dosage of the active substances can vary depending on the route of administration, the patient's age and weight, the nature and severity of the disease to be treated and similar factors. The daily dose is 0.5-1000 mg, preferably 50-200 mg, and the dose can be a single dose that is to be administered once, or can be divided into 2 or more daily doses.

Inhibitors of soluble adenylate cyclase lead to depression of the cAMP level. The cAMP level is decisive for control of the processes that play an important role in cell proliferation, cell differentiation and apoptosis. Diseases, e.g. cancer, in which depression of the cAMP level is decisive, can be modulated by inhibitors of soluble adenylate cyclase. This modulation can have prophylactic and therapeutic effects for patients suffering such a disease. At the present time diseases which are, like cancer, associated with increased cell proliferation, are treated, for example by radiotherapy and chemotherapy. These methods are nonspecific and have a high potential for side effects. The provision of new substances, which act directly on particular target sites, is therefore advantageous. The present invention relates to substances that modulate cAMP production by the inhibition of soluble adenylate cyclase. For example, abnormal cell proliferation can be reduced or prevented by regulation or inhibition of cAMP production. Soluble adenylate cyclase can be inhibited by the use of the substances according to the invention, with a consequent reduction in cell proliferation.

The present invention relates to substances according to general formula I, that reduce or inhibit the activity of the soluble adenylate cyclase.

The present invention also relates to medicinal products that contain at least one compound according to general formula I, for the treatment of diseases which are characterized in that they are caused by disturbances of metabolism of the second messenger cAMP.

The use of the substances according to the invention leads to an inhibition of the soluble adenylate cyclase and therefore to a lowering of the cAMP concentration and, consequently, to a reduction or inhibition of sperm capacitation.

The present invention relates to the use of the substances according to the invention for the lowering and/or inhibition of male gamete fertility mediated by the reduction or inhibition of soluble adenylate cyclase activity and accordingly of sperm capacitation.

Fertilization of the ovum is prevented by administering an effective amount of a substance that leads to inhibition of cAMP production.

The present invention also relates to the use of the compound of general formula I for the production of a contraceptive.

The present invention also relates to a suppository, characterized in that it contains at least one of the compounds according to formula I and is used, for example, for female contraception.

If the production of the starting compounds is not described, these are known or can be produced similarly to known compounds or methods described here. It is also possible to carry out all the reactions described here in parallel reactors or using combinatorial techniques.

The mixtures of isomers can be separated into the enantiomers or E/Z isomers by usual methods, for example crystallization, chromatography or salt formation.

The salts are produced in the usual manner, by adding the equivalent amount or an excess of a base or acid, which is in solution if necessary, to a solution of the compound of formula I, separating the precipitate or processing the solution in the usual way.

### Production of the Compounds According to the Invention

The following examples explain the production of the compounds of general formula I according to the invention, without limiting the scope of the claimed compounds to these examples.

The compounds of general formula I according to the invention can be produced as described below.

### EXAMPLE 1

### N-(Tetrahydropyran-4-yl)-5-(4-tert-butylphenylsulfonylamino)-3-phenyl-1H-pyrrolo[3,2-b]pyridine-2-carboxamide

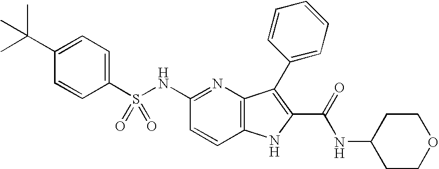

A solution of 50 mg of the acid prepared in example 1j) in 0.90 ml of dimethylformamide is admixed with 46.1 mg of N-[(dimethylamino)-1H-1,2,3-triazolo[4,5-b]pyridin-1-ylmethylene]-N-methylmethanaminium hexafluoro-phosphate N-oxide (HATU) and 11.3 mg of 4-aminotetrahydropyran. Then, at 0° C., 20.7 μl of ethyldiisopropylamine are added dropwise and the mixture is stirred at room temperature for 20 hours. Subsequently 25 ml of water are added and the mixture is stirred for 30 minutes and filtered with suction over a G4 frit. The resulting residue is purified by chromatography on silica gel using hexane/0-90% ethyl acetate. This gives 38 mg of the title compound.

NMR (300 MHz, DMSO-d6): δ=1.20 (9H), 1.27 (2H), 1.67 (2H), 3.33 (2H), 3.69 (2H), 3.91 (1H), 6.91 (1H), 7.26-7.58 (8H), 7.72 (1H), 7.75 (2H), 10.72 (1H), 11.82 (1H).

The starting material for the above title compound is prepared as follows:

### 1a) 6-Methyl-5-nitropyridin-2-ylamine

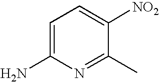

A solution of 211 g of 6-amino-2-picoline in 994 ml of concentrated sulfuric acid is admixed dropwise at 0° C. with a cooled mixture of 145 ml of concentrated nitric acid and 145 ml of concentrated sulfuric acid. The reaction mixture is stirred at 0° C. for one hour and then at 25° C. for 16 hours. Subsequently it is heated at 60° C. for one hour and finally at 100° C. for one hour. The reaction mixture is then poured cautiously onto ice and adjusted to a pH of 5 to 6 with a concentrated sodium hydroxide solution. The resultant solid is filtered off and washed with ethanol. The resulting crude product is prepurified by column chromatography on silica gel with chloroform/0-50% methanol. The material is then partly dissolved in 100 ml of 1N hydrochloric acid, the precipitate is removed by filtration, and the solution is admixed with sodium carbonate. The resulting solid is recovered by filtration. Drying gives 33 g of the title compound as a yellow-orange solid.

NMR (300 MHz, DMSO-d6): δ=2.60 (3H), 6.37 (1H), 7.35 (2H), 8.09 (1H).

### 1b) 6-Methyl-5-nitro-2-(N,N-di-tert-butyloxycarbonylamino)pyridine

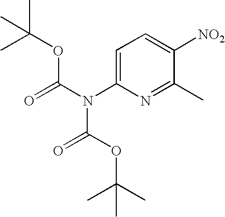

A solution of 24.3 g of the above-prepared nitro compound and 0.35 g of DMAP in 500 ml of methylene chloride is admixed with a solution of 69 g of Boc₂O in 500 ml of methylene chloride and stirred at 25° C. for 16 hours. The organic phase is subsequently washed with three times 700 ml of water and dried over sodium sulfate. Filtration gives, without further purification, 49 g of the title compound as a yellow solid.

NMR (300 MHz, DMSO-d6): δ=1.46 (18H), 2.69 (3H), 7.63 (1H), 8.51 (1H).

### 1c) Ethyl 3-(6-tert-butoxycarbonylamino-3-nitropyridin-2-yl)-2-oxopropionate

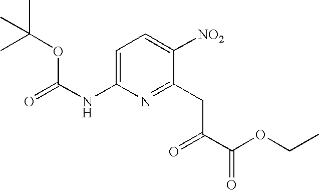

A solution of sodium ethoxide in ethanol (prepared by dissolving 6.7 g of sodium in 490 ml of ethanol) is admixed with 49 g of the above-prepared picoline derivative. After 10 minutes of stirring, 42.6 g of diethyl oxalate are added in one portion and the mixture is stirred at 25° C. for 5 days. Following addition of 1000 ml of ether, the precipitate formed is isolated by filtration, then suspended in water and acidified to a pH of 4 with 1N hydrochloric acid. The solid is isolated by filtration, washed with water and dried. This gives 18 g of the title compound.

NMR (300 MHz, DMSO-d6): δ=1.29 (3H), 1.51 (9H), 4.28 (2H), 7.30 (1H), 7.37 (1H), 8.51 (1H), 11.06 (1H), 13.42 (1H).

### 1d) Ethyl 5-tert-butoxycarbonylamino-1H-pyrrolo[3,2-b]pyridine-2-carboxylate

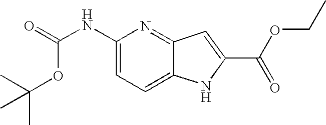

18 g of the ester prepared in example 1c) are dissolved in 500 ml of ethanol and hydrogenated with 3 g of palladium on carbon in an autoclave at a pressure of 20 atm hydrogen and a temperature of 50° C. for 6 hours. Following filtration and concentration under reduced pressure, the resulting crude product is purified by column chromatography on silica gel with chloroform/0-10% ethyl acetate. This gives 4.2 g of the title compound.

NMR (300 MHz, DMSO-d6): δ=1.34 (3H), 1.46 (9H), 4.35 (2H), 6.99 (1H), 7.78 (2H), 9.63 (1H), 12.03 (1H).

### 1e) Ethyl 5-amino-1H-pyrrolo[3,2-b]pyridine-2-carboxylate

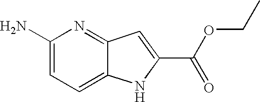

4.2 g of the compound prepared in example 1d) are admixed with 30 ml of a 4 M solution of HCl in dioxane and the mixture is stirred at 25° C. for 16 hours. Following filtration and concentration under reduced pressure, the residue is dissolved in 30 ml of water and extracted with 50 ml of ether. The aqueous phase is then adjusted to a pH of 10 to 11 with saturated sodium carbonate solution and extracted with a mixture of chloroform and methanol in a ratio of 10:1. The combined organic phases are washed with saturated sodium chloride solution, dried over sodium sulfate, filtered and concentrated under reduced pressure. This gives 2.7 g of the title compound.

NMR (300 MHz, DMSO-d6): δ=1.31 (3H), 4.30 (2H), 5.68 (2H), 6.52 (1H), 6.73 (2H), 7.58 (1H), 11.59 (1H).

### 1f) Ethyl 5-(4-tert-butylphenylsulfonylamino)-1H-pyrrolo[3,2-b]pyridine-2-carboxylate

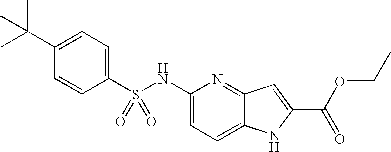

A solution of 2 g of the amine prepared in example if) in 97 ml of pyridine is admixed at 25° C. with 2.27 g of 4-tert-butylphenylsulfonyl chloride and the mixture is stirred at this temperature for 20 hours. With addition of toluene, the solvent is removed under reduced pressure and the residue is purified by chromatography on silica gel with hexane/0-80% ethyl acetate. This gives 3.3 g of the title compound.

NMR (300 MHz, DMSO-d6): δ=1.21 (9H), 1.29 (3H), 4.29 (2H), 6.88 (1H), 7.13 (1H), 7.52 (2H), 7.76 (1H), 7.80 (2H), 11.38 (1H), 12.17 (1H).

### 1g) Ethyl 3-bromo-5-(4-tert-butylphenylsulfonylamino)-1H-pyrrolo[3,2-b]pyridine-2-carboxylate

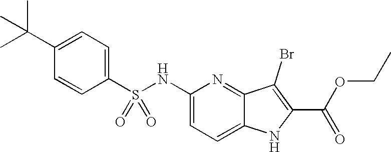

A solution of 500 mg of the sulfonamide prepared in example if) in 14 ml of tetrahydrofuran is admixed with 222 mg of N-bromosuccinimide and the mixture is stirred at 25° C. for 60 minutes. It is diluted with 200 ml of ethyl acetate and washed with twice 20 ml of half-concentrated sodium chloride solution and the organic phase is dried over sodium sulfate. Filtration and concentration under reduced pressure are followed by purification of the resultant residue by chromatography on silica gel with hexane/0-50% ethyl acetate. This gives 585 mg of the title compound.

NMR (300 MHz, DMSO-d6): δ=1.22 (9H), 1.32 (3H), 4.33 (2H), 7.02 (1H), 7.55 (2H), 7.76 (1H), 8.02 (2H), 11.01 (1H), 12.33 (1H).

### 1h) Ethyl 1-tert-butoxycarbonyl-3-bromo-5-[(tert-butoxycarbonyl)-(4-tert-butyl-phenylsulfonyl)amino]-1H-pyrrolo[3,2-b]pyridine-2-carboxylate

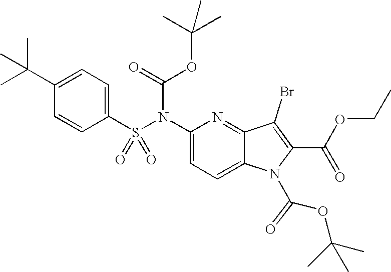

A solution of 638 mg of the bromide prepared in example 1g) in 6.6 ml of methylene chloride is admixed with 81 mg of DMAP and 1.16 g of di-tert-butyl dicarbonate and the mixture is stirred at 25° C. for 3 days. Following concentration under reduced pressure, the resulting residue is purified by chromatography on silica gel with hexane/0-50% ethyl acetate. This gives 715 mg of the title compound.

NMR (300 MHz, DMSO-d6): δ=1.20 (9H), 1.31 (9H), 1.33 (3H), 1.58 (9H), 4.41 (2H), 7.69 (1H), 7.72 (2H), 8.09 (2H), 8.47 (1H).

### 1i) Ethyl 3-phenyl-5-(4-tert-butylphenylsulfonylamino)-1H-pyrrolo[3,2-b]pyridine-2-carboxylate

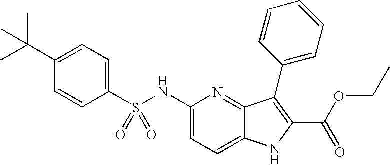

A solution of 350 mg of the protected bromide prepared in example 1h) in 11.2 ml of dioxane is admixed with 627 mg of phenylboronic acid and 1.09 g of potassium phosphate. Following addition of 75.3 mg of 1,1′-bis(diphenyl-phosphino)ferrocenedichloropalladium(II) the reaction mixture is heated at 90° C. for 20 hours. After cooling it is diluted with 200 ml of ethyl acetate and the organic phase is washed with twice 20 ml of a half-concentrated sodium chloride solution and dried over sodium sulfate. Following filtration and concentration under reduced pressure, the resulting residue is purified by chromatography on silica gel with hexane/0-50% ethyl acetate. This gives 214 mg of ethyl 1-tert-butoxycarbonyl-3-phenyl-5-[(tert-butoxycarbonyl)-(4-tert-butylphenylsulfonyl)amino]-1H-pyrrolo[3,2-b]pyridine-2-carboxylate. 210 mg of this product are admixed with 5 ml of trifluoroacetic acid and stirred at 25° C. for 20 hours. Following addition of toluene the mixture is concentrated under reduced pressure. The resulting residue is purified by chromatography on silica gel with hexane/0-50% ethyl acetate. This gives 135 mg of the title compound.

NMR (300 MHz, DMSO-d6): δ=1.14 (3H), 1.19 (9H), 4.18 (2H), 6.97 (1H), 7.31-7.44 (7H), 7.72 (2H), 7.77 (1H), 10.79 (1H), 11.99 (1H).

### 1j) 3-Phenyl-5-(4-tert-butylphenylsulfonylamino)-1H-pyrrolo[3,2-b]pyridine-2-carboxylic acid

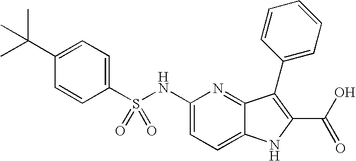

A mixture of 130 mg of the ester prepared in example 1i) in 3.2 ml of ethanol and 2 ml of tetrahydrofuran is admixed with 210 mg of sodium hydroxide in solution in 1.6 ml of water and the mixture is stirred at 25° C. for 20 hours. Subsequently it is diluted with 100 ml of water and acidified with 5% strength aqueous sulfuric acid. The precipitate is isolated by filtration and dried. This gives 122 mg of the title compound, which is reacted further without further purification.

NMR (300 MHz, DMSO-d6): δ=1.19 (9H), 6.96 (1H), 7.30-7.45 (7H), 7.71 (2H), 7.74 (1H), 10.76 (1H), 11.90 (1H), 13.02 (1H).

### EXAMPLE 2

### N-(2-Morpholin-4-ylethyl)-5-(4-tert-butylphenylsulfonylamino)-3-phenyl-1H-pyrrolo[3,2-b]pyridine-2-carboxamide

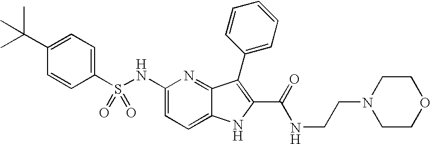

In analogy to example 1, from 50 mg of the acid from example 1j) and 14.5 mg of 2-(morpholin-4-yl)ethylamine, 30 mg of the title compound are obtained.

NMR (300 MHz, DMSO-d6): δ=1.25 (9H), 2.21-2.39 (6H), 3.30 (2H), 3.44 (4H), 6.96 (1H), 7.21 (1H), 7.39-7.52 (7H), 7.76 (3H), 10.76 (1H), 11.86 (1H).

### EXAMPLE 3

### N-(2-Morpholin-4-ylethyl)-5-(4-tert-butylphenylsulfonylamino)-3-phenyl-1H-pyrrolo[2,3-c]pyridine-2-carboxamide

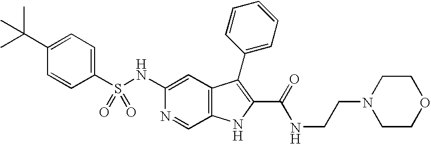

In analogy to example 1), from 108 mg of the acid prepared in example 31) and 31.4 mg of 2-(morpholin-4-yl)ethylamine, 54 mg of the title compound are obtained.

NMR (300 MHz, DMSO-d6): δ=1.21 (9H), 2.16-2.31 (6H), 3.27 (2H), 3.40 (4H), 7.19 (1H), 7.34-7.41 (3H), 7.43 (1H), 7.46-7.56 (4H), 7.67 (2H), 8.41 (1H), 10.48 (1H), 12.13 (1H).

The starting material for the above title compound is prepared as follows:

### 3a) 4-Methyl-5-nitro-2-(N,N-di-tert-butyloxycarbonylamino)pyridine

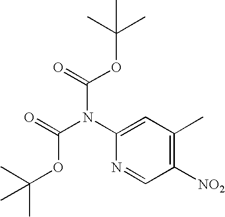

A solution of 1.0 g of 2-amino-4-methyl-5-nitropyridine and 15 mg of DMAP in 20 ml of methylene chloride is admixed with a solution of 2.85 g of Boc₂O in 10 ml of methylene chloride and the mixture is stirred at 25° C. for 16 hours. Subsequently the organic phase is washed with three times 50 ml of water and dried over sodium sulfate. Without further purification, filtration gives 2.2 g of the title compound as a dark yellow solid.

NMR (300 MHz, DMSO-d6): δ=1.45 (18H), 2.62 (3H), 7.63 (1H), 9.04 (1H).

### 3b) Ethyl 3-(6-tert-butoxycarbonylamino-3-nitropyridin-4-yl)-2-oxopropionate

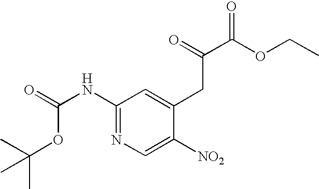

A solution of sodium ethoxide in ethanol (prepared by dissolving 10.6 g of sodium in 770 ml of ethanol) is admixed with 77 g of the pyridine derivative prepared in example 3a). After 5 minutes of stirring, 67.3 g of diethyl oxalate are added in one portion and the mixture is stirred at 25° C. for 7 days. During this time a precipitate is formed, which is isolated by filtration and washed with 20 ml of ethanol. The precipitate is suspended in the minimum amount of water and the pH is adjusted to 4 by means of 1-molar hydrochloric acid. After repeated filtration, the product is washed with a little water and, after brief drying, with hexane. Drying in air gives 53.0 g of the title compound as a yellow-orange solid.

NMR (300 MHz, DMSO-d6): δ=1.24 (3H), 1.48 (9H), 4.93 (2H), 6.64 (1H), 8.66 (1H), 9.32 (1H), 9.43 (1H).

### 3c) Ethyl 5-tert-butoxycarbonylamino-1H-pyrrolo[2,3-c]pyridine-2-carboxylate

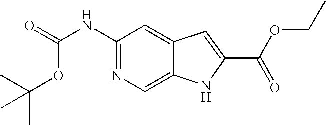

52 g of the ester prepared in example 3b) are dissolved in 550 ml of ethanol and hydrogenated with 6 g of palladium on carbon in an autoclave at a pressure of 40-45 atm hydrogen and a temperature of 50° C. for 5 hours. Filtration and concentration under reduced pressure give 21.5 g of the title compound, which is reacted further without further purification.

NMR (300 MHz, DMSO-d6): δ=1.15 (3H), 1.46 (9H), 4.35 (2H), 7.09 (1H), 7.95 (1H), 8.54 (1H), 9.44 (1H).

### 3d) Ethyl 5-amino-1H-pyrrolo[2,3-c]pyridine-2-carboxylate

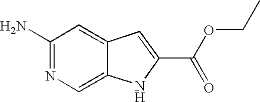

21 g of the compound prepared in example 3c) are admixed with 40 ml of a 4 M solution of HCl in dioxane and the mixture is stirred at 25° C. for 3 hours. After filtration and concentration under reduced pressure the residue is dissolved in 100 ml of water. The aqueous phase is adjusted to a pH of 10 to 11 with saturated sodium carbonate solution and extracted with a mixture of chloroform and t-butanol in a ratio of 8:1. The combined organic phases are washed with saturated sodium chloride solution, dried over sodium sulfate, filtered and concentrated under reduced pressure. This gives 7.5 g of the title compound.

NMR (300 MHz, DMSO-d6): δ=1.32 (3H), 4.32 (2H), 5.17 (1H), 6.57 (1H), 6.83 (1H), 8.32 (1H), 11.70 (1H).

### 3e) Ethyl 5-(4-tert-butylphenylsulfonylamino)-1H-pyrrolo[2,3-c]pyridine-2-carboxylate

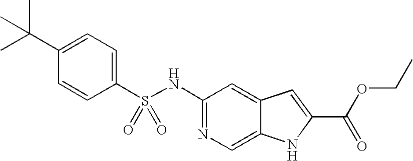

In analogy to example 1f), from 1.93 g of the amine prepared in example 3d), 3.2 g of the title compound are obtained.

NMR (300 MHz, DMSO-d6): δ=1.20 (9H), 1.29 (3H), 4.32 (2H), 7.08 (1H), 7.41 (1H), 7.50 (2H), 7.72 (2H), 8.43 (1H), 10.49 (1H), 12.23 (1H).

### 3f) Ethyl 3-bromo-5-(4-tert-butylphenylsulfonylamino)-1H-pyrrolo[2,3-c]pyridine-2-carboxylate

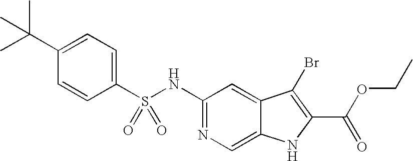

In analogy to example 1g), from 3.2 g of the ester prepared in example 3e), 1.48 g of the title compound are obtained.

NMR (300 MHz, 300 MHz, DMSO-d6): δ=1.20 (9H), 1.32 (3H), 4.35 (2H), 7.22 (1H), 7.52 (2H), 7.73 (2H), 8.47 (1H), 10.70 (1H), 12.60 (1H).

### 3g) Ethyl 1-tert-butoxycarbonyl-3-bromo-5-[(tert-butoxycarbonyl)-(4-tert-butyl-phenylsulfonyl)amino]-1H-pyrrolo[2,3-c]pyridine-2-carboxylate

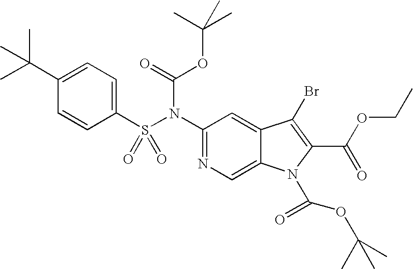

In analogy to example 1h), from 1.48 g of the bromide prepared in example 3f), 1.68 g of the title compound are obtained.

NMR (300 MHz, DMSO-d6): δ=1.17 (9H), 1.31 (9H), 1.33 (3H), 1.61 (9H), 4.41 (2H), 7.71 (2H), 7.77 (1H), 8.03 (2H), 9.21 (1H).

### 3h) Ethyl 3-phenyl-5-(4-tert-butylphenylsulfonylamino)-1H-pyrrolo[2,3-c]pyridine-2-carboxylate

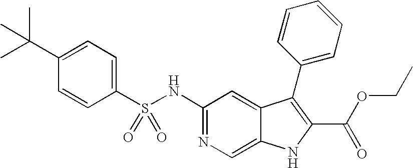

In analogy to example 1i), from 300 mg of the bromide prepared in example 3g), 137 mg of the title compound are obtained.

NMR (300 MHz, DMSO-d6): δ=1.14 (3H), 1.21 (9H), 4.20 (2H), 7.18 (1H), 7.34-7.41 (3H), 7.44 (2H), 7.52 (2H), 7.68 (2H), 8.50 (1H), 10.51 (1H), 11.29 (1H).

### 3i) 3-Phenyl-5-(4-tert-butylphenylsulfonylamino)-1H-pyrrolo[2,3-c]pyridine-2-carboxylic acid

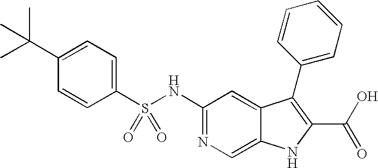

In analogy to example 1j), from 120 mg of the ester prepared in example 3h), 112 mg of the title compound are obtained.

NMR (300 MHz, DMSO-d6): δ=1.21 (9H), 7.16 (1H), 7.32-7.47 (6H), 7.52 (2H), 7.66 (2H), 8.47 (1H), 10.54 (1H), 12.22 (1H).

### EXAMPLE 4

### N-(2-Morpholin-4-ylethyl)-5-(4-tert-butylphenylsulfonylamino)-3-phenyl-1H-pyrrolo[2,3-b]pyridine-2-carboxamide

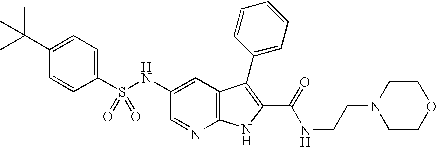

In analogy to example 1), from 100 mg of the acid prepared in example 4h), and 29 mg of 2-(morpholin-4-yl)ethylamine, 63 mg of the title compound are obtained.

NMR (300 MHz, DMSO-d6): δ=1.26 (9H), 2.24-2.36 (6H), 3.30 (2H), 3.46 (4H), 7.30-7.53 (7H), 7.57 (4H), 8.08 (1H), 10.01 (1H), 12.32 (1H).

The starting material for the above title compound is prepared as follows:

### 4a) 2-Hydroxy-5-nitronicotinic acid

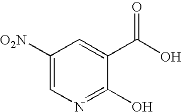

A solution of 210 g of 2-hydroxynicotinic acid in 1.5 l of concentrated sulfuric acid is cooled to −5° C. and then admixed dropwise with 100 ml of concentrated nitric acid. Subsequently the mixture is left to warm to 25° C. and is stirred at that temperature for 16 hours. Then it is heated at 50° C. for 1 hour and, after cooling, the reaction mixture is poured cautiously onto 3 kg of ice. The white precipitate is isolated by filtration, washed once with water, and recrystallized from a water/ethanol mixture. This gives 190 g of the title compound.

NMR (300 MHz, DMSO-d6): δ=8.7 (1H), 9.0 (1H).

### 4b) 2-Chloro-5-nitropyridin-3-yl phenyl ketone

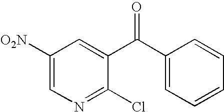

18.4 g of the acid prepared in example 4a) is admixed at 25° C. with 120 ml of thionyl chloride followed by 3 ml of DMF, and subsequently heated at reflux for 3 hours. The excess thionyl chloride is removed under reduced pressure and the yellow solid that remains is dissolved in 100 ml of benzene. This solution is admixed with 40 g of AlCl₃ at 0° C. and then stirred at 25° C. for 16 hours. The reaction mixture is then poured cautiously into cold (partly frozen) concentrated hydrochloric acid; after one hour the mixture is filtered and the organic phase is separated off. The organic phase is washed with hydrochloric acid, dried over sodium sulfate, filtered and concentrated under reduced pressure. The resulting residue is purified by chromatography on silica gel with hexane/10% ethyl acetate. This gives 15 g of the title compound.

NMR (300 MHz, DMSO-d6): δ=7.60 (2H), 7.77 (1H), 7.87 (2H), 8.95 (1H), 9.4 (1H).

### 4c) Ethyl (3-benzoyl-5-nitropyridin-2-yl)-(4-methoxybenzyl)amino]acetate

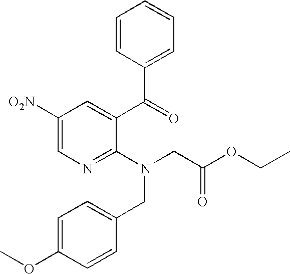

A solution of 18.4 g of the ketone prepared in example 4b) in 150 ml of acetonitrile is admixed with 23.1 g of N-(4-methoxybenzyl)glycine ethyl ester followed by a solution of 13 g of diisopropylethylamine in 100 ml of acetonitrile. After 16 hours of stirring at 25° C., all of the volatile constituents are removed under reduced pressure and the residue is dissolved in ethyl acetate. This solution is washed with three times 100 ml of water, dried over sodium sulfate, filtered and concentrated under reduced pressure. Recrystallization gives 32 g of the title compound as a white solid.

NMR (300 MHz, DMSO-d6): δ=1.1 (3H), 3.7 (3H), 4.0 (2H), 4.3 (2H), 4.7 (2H), 6.7 (2H), 7.0 (2H), 7.5 (2H), 7.7 (2H), 7.7 (1H), 8.2 (1H), 9.1 (1H).

### 4d) Ethyl 1-(4-methoxybenzyl)-5-nitro-3-phenyl-1H-pyrrolo[2,3-b]pyridine-2-carboxylate

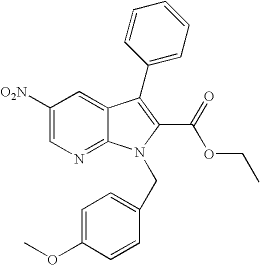

A solution of 32 g of the ester prepared in example 4c) in 50 ml of pyridine is admixed under argon with 100 ml of acetic anhydride and the mixture is subsequently stirred at 100° C. for 16 hours. Then all of the volatile constituents are removed under reduced pressure and the resulting residue is purified by chromatography on silica gel with methylene chloride. This gives 24.5 g of the title compound.

NMR (300 MHz, DMSO-d6): δ=1.0 (3H), 3.7 (3H), 4.2 (2H), 5.9 (2H), 6.9 (2H), 7.1 (2H), 7.5 (5H), 8.7 (1H), 9.4 (1H).

### 4e) Ethyl 5-nitro-3-phenyl-1H-pyrrolo[2,3-b]pyridine-2-carboxylate

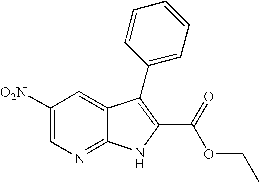

A solution of 27.2 g of the indole derivative prepared in example 4d) in 150 ml of trifluoroacetic acid is admixed with 2.5 ml of anisole and this mixture is heated at reflux for 3 days. Subsequently all of the volatile constituents are removed under reduced pressure and the resulting residue is purified by recrystallization from ethyl acetate. This gives 20 g of the title compound.

NMR (300 MHz, DMSO-d6): δ=1.2 (3H), 4.3 (2H), 7.5 (5H), 8.6 (1H), 9.3 (1H), 13.4 (1H).

### 4f) Ethyl 5-amino-3-phenyl-1H-pyrrolo[2,3-b]pyridine-2-carboxylate hydrochloride

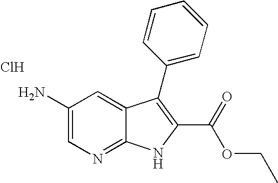

18 g of the nitro indole prepared in example 4e) are dissolved in 500 ml of dioxane and hydrogenated with 4 g of palladium on carbon in an autoclave at a pressure of 50 psi hydrogen at 25° C. for 3 days. Filtration and concentration under reduced pressure to a volume of approximately 200 ml are followed by the addition to this solution of 50 ml of a 7 M solution of HCl in dioxane. The precipitate formed is isolated by filtration, washed with dioxane and dried. This gives 16 g of the title compound as a white solid.

NMR (300 MHz, DMSO-d6): δ=1.2 (3H), 4.2 (2H), 7.5 (5H), 8.0 (1H), 8.5 (1H), 10.4 (1H), 12.9 (1H).

### 4g) Ethyl 5-(4-tert-butylphenylsulfonylamino)-3-phenyl-1H-pyrrolo[2,3-b]pyridine-2-carboxylate

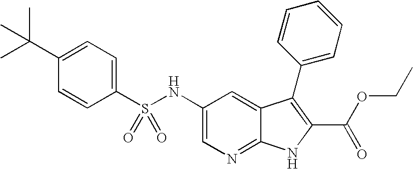

In analogy to example 1f), from 2.0 g of the amine prepared in example 4f), 2.31 g of the title compound are obtained.

NMR (300 MHz, DMSO-d6): δ=1.16 (3H), 1.26 (9H), 4.21 (2H), 7.29-7.35 (2H), 7.38-7.48 (4H), 7.57 (4H), 8.18 (1H), 10.07 (1H), 12.60 (1H).

### 4h) 3-Phenyl-5-(4-tert-butylphenylsulfonylamino)-1H-pyrrolo[2,3-b]pyridine-2-carboxylic acid

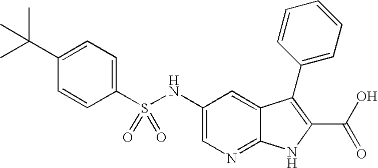

In analogy to example 1j), from 2.3 g of the ester prepared in example 4g), 2.16 g of the title compound are obtained.

NMR (300 MHz, DMSO-d6): δ=1.21 (9H), 7.26-7.42 (6H), 7.52 (4H), 8.10 (1H), 9.99 (1H), 12.34 (1H), 13.12 (1H).

### EXAMPLE 5

### N-(Tetrahydropyran-4-yl)-5-(4-tert-butylphenylsulfonylamino)-3-phenyl-1H-pyrrolo[2,3-b]pyridine-2-carboxamide

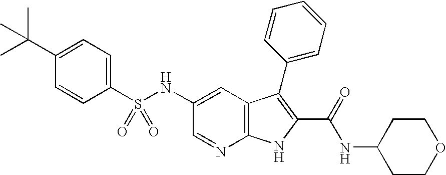

In analogy to example 1), from 100 mg of the acid prepared in example 4h) and 23 mg of 4-aminotetrahydropyran, 49 mg of the title compound are obtained.

NMR (300 MHz, DMSO-d6): δ=1.26 (9H), 1.35 (2H), 1.71 (2H), 3.35 (2H), 3.77 (2H), 3.93 (1H), 7.28-7.52 (6H), 7.57 (4H), 7.86 (1H), 8.07 (1H), 10.02 (1H), 12.32 (1H).

### EXAMPLE 6

### (±)-N-(2-Hydroxy-1-methylethyl)-5-(4-tert-butylphenylsulfonyl-amino)-3-phenyl-1H-pyrrolo[2,3-b]pyridine-2-carboxamide

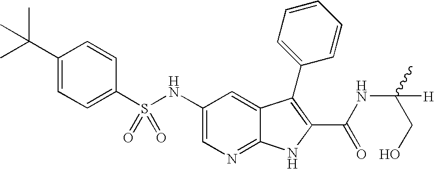

In analogy to example 1), from 100 mg of the acid prepared in example 4h) and 17.7 μl of (±)-2-aminopropan-1-ol, 75 mg of the title compound are obtained.

NMR (300 MHz, DMSO-d6): δ=0.97 (3H), 1.21 (9H), 3.18 (1H), 3.30 (1H), 3.89 (1H), 4.60 (1H), 7.23-7.45 (6H), 7.49-7.57 (6H), 8.03 (1H), 9.96 (1H), 12.23 (1H).

### EXAMPLE 7

### (±)-N-(2-Hydroxypropyl)-5-(4-tert-butylphenylsulfonylamino)-3-phenyl-1H-pyrrolo[2,3-b]pyridine-2-carboxamide

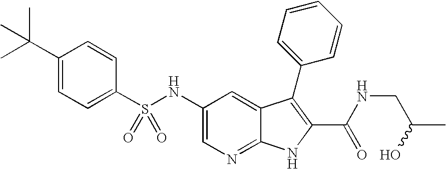

In analogy to example 1), from 100 mg of the acid prepared in example 4h) and 17.6 μl of (±)-2-hydroxypropylamine, 88 mg of the title compound are obtained.

NMR (300 MHz, DMSO-d6): δ=0.93 (3H), 1.21 (9H), 3.18 (1H), 3.08 (2H), 3.61 (1H), 4.60 (1H), 7.23-7.44 (6H), 7.52 (4H), 7.71 (1H), 8.04 (1H), 9.96 (1H), 12.23 (1H).

### EXAMPLE 8

### (1S,2R)—N-(2-Hydroxycyclopentyl)-5-(4-tert-butylphenylsulfonyl-amino)-3-phenyl-1H-pyrrolo[2,3-b]pyridine-2-carboxamide

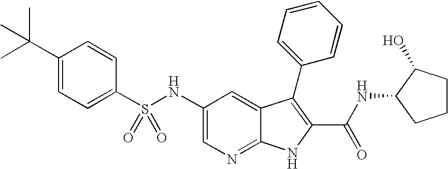

In analogy to example 1), from 100 mg of the acid prepared in example 4h) and 30.6 mg of (1S,2R)-2-hydroxycyclopentylamine, 88 mg of the title compound are obtained.

NMR (300 MHz, DMSO-d6): δ=1.21 (9H), 1.32-1.80 (6H), 3.87 (1H), 3.95 (1H), 4.52 (1H), 7.11 (1H), 7.24-7.44 (6H), 7.51 (4H), 8.05 (1H), 9.96 (1H), 12.29 (1H).

### EXAMPLE 9

### N-(Pyridin-3-ylmethyl)-5-(4-tert-butylphenylsulfonylamino)-3-phenyl-1H-pyrrolo[2,3-b]pyridine-2-carboxamide

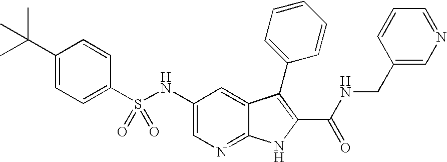

In analogy to example 1), from 70 mg of the acid prepared in example 4h) and 15.9 μl of 3-pyridylmethylamine, 58 mg of the title compound are obtained.

NMR (300 MHz, DMSO-d6): δ=1.25 (9H), 4.41 (2H), 7.24 (1H), 7.26 (1H), 7.29-7.43 (4H), 7.48 (1H), 7.57 (4H), 7.65 (1H), 8.08 (1H), 8.46 (1H), 8.50 (1H), 8.59 (1H), 10.03 (1H), 12.37 (1H).

### EXAMPLE 10

### (±)-N-(Tetrahydrofuran-2-ylmethyl)-5-(4-tert-butylphenylsulfonyl-amino)-3-phenyl-1H-pyrrolo[2,3-b]pyridine-2-carboxamide

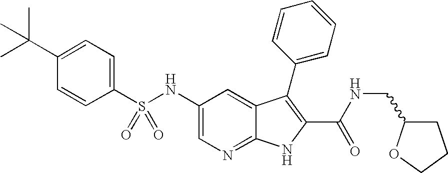

In analogy to example 1), from 100 mg of the acid prepared in example 4h) and 22.5 mg of (tetrahydrofuran-2-yl)methylamine, 92 mg of the title compound are obtained.

NMR (300 MHz, DMSO-d6): δ=1.21 (9H), 1.40 (1H), 1.64-1.84 (3H), 3.10-3.29 (2H), 3.52 (1H), 3.61 (1H), 3.79 (1H), 7.23-7.44 (6H), 7.52 (4H), 7.70 (1H), 8.03 (1H), 8.96 (1H), 12.25 (1H).

### EXAMPLE 11

### N-[2-(Morpholine-4-sulfonyl)ethyl]-5-(4-tert-butylphenylsulfonyl-amino)-3-phenyl-1H-pyrrolo[2,3-b]pyridine-2-carboxamide

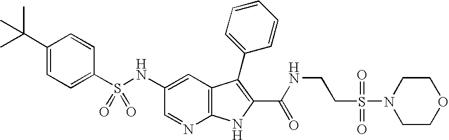

In analogy to example 1), from 70 mg of the acid prepared in example 4h) and 35.9 mg of 2-(morpholine-4-sulfonyl)ethylamine, 58 mg of the title compound are obtained.

NMR (300 MHz, DMSO-d6): δ=1.26 (9H), 3.14 (4H), 3.21 (2H), 3.58 (2H), 3.64 (4H), 7.29-7.51 (6H), 7.57 (4H), 8.06 (1H), 8.10 (1H), 10.04 (1H), 12.32 (1H).

### EXAMPLE 12

### N-(pyridin-4-yl)-5-(4-tert-butylphenylsulfonylamino)-3-phenyl-1H-pyrrolo[2,3-b]pyridine-2-carboxamide

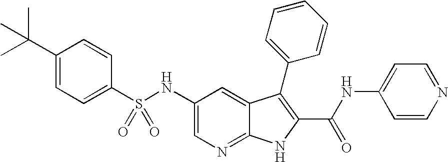

In analogy to example 1), from 70 mg of the acid prepared in example 4h) and 14.7 mg of 4-aminopyridine, 25 mg of the title compound are obtained.

NMR (300 MHz, DMSO-d6): δ=1.26 (9H), 7.32-7.63 (13H), 8.15 (1H), 8.46 (2H), 10.10 (1H), 10.51 (1H), 12.60 (1H).

### EXAMPLE 13

### N-[2-(2-Oxoimidazolidin-1-yl)ethyl]-5-(4-tert-butylphenylsulfonyl-amino)-3-phenyl-1H-pyrrolo[2,3-b]pyridine-2-carboxamide

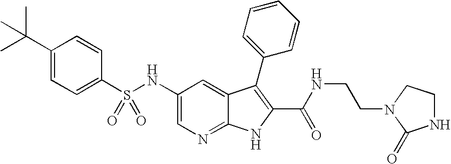

In analogy to example 1), from 70 mg of the acid prepared in example 4h) and 40.2 mg of 1-(2-aminoethyl)imidazolidin-2-one, 57 mg of the title compound are obtained.

NMR (300 MHz, DMSO-d6): δ=1.21 (9H), 3.12 (4H), 3.25 (4H), 6.30 (1H), 7.22-7.45 (6H), 7.52 (4H), 7.93 (1H), 8.03 (1H), 9.96 (1H), 12.15 (1H).

### EXAMPLE 14

### N-Ethyl-5-(4-tert-butylphenylsulfonylamino)-3-phenyl-1H-pyrrolo-[2,3-b]pyridine-2-carboxamide

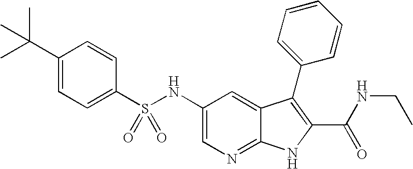

In analogy to example 1), from 70 mg of the acid prepared in example 4h) and 80 μl of ethylamine, 42 mg of the title compound are obtained.

NMR (300 MHz, DMSO-d6): δ=0.98 (3H), 1.21 (9H), 3.15 (2H), 7.22-7.46 (6H), 7.52 (4H), 7.87 (1H), 8.02 (1H), 9.96 (1H), 12.22 (1H).

### EXAMPLE 15

### 5-(4-tert-Butylphenylsulfonylamino)-3-phenyl-1H-pyrrolo[2,3-b]-pyridine-2-carboxamide

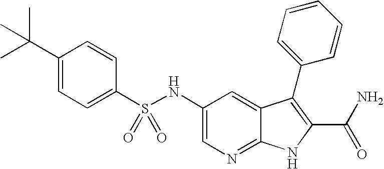

In analogy to example 1), from 70 mg of the acid prepared in example 4h) and 75 μl of 2M NH₃ solution in methanol, 40 mg of the title compound are obtained.

NMR (300 MHz, DMSO-d6): δ=1.26 (9H), 7.19 (2H), 7.28-7.49 (6H), 7.56 (4H), 8.08 (1H), 10.01 (1H), 12.23 (1H).

### EXAMPLE 16

### N-(2-Sulfamoylethyl)-5-(4-tert-butylphenylsulfonylamino)-3-phenyl-1H-pyrrolo[2,3-b]pyridine-2-carboxamide

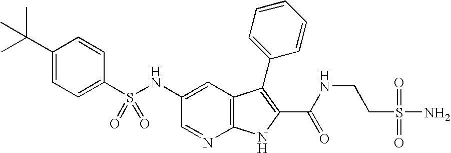

In analogy to example 1), from 70 mg of the acid prepared in example 4h) and 19.3 mg of 2-aminoethanesulfonamide, 60 mg of the title compound are obtained.

NMR (300 MHz, DMSO-d6): δ=1.26 (9H), 3.14 (2H), 3.58 (2H), 6.94 (2H), 7.27-7.51 (6H), 7.57 (4H), 8.09 (1H), 8.13 (1H), 10.03 (1H), 12.29 (1H).

### EXAMPLE 17

### N-(2-Morpholin-4-ylethyl)-5-(biphenyl-4-sulfonylamino)-3-phenyl-1H-pyrrolo[2,3-b]pyridine-2-carboxamide

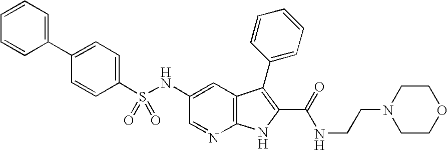

In analogy to example 1), from 50 mg of the acid prepared in example 17b) and 13.9 mg of 2-(morpholin-4-yl)ethylamine, 15 mg of the title compound are obtained.

NMR (300 MHz, DMSO-d6): δ=2.27 (4H), 2.32 (2H), 3.30 (2H), 3.45 (4H), 7.27-7.39 (5H), 7.42 (1H), 7.48 (2H), 7.52 (2H), 7.71 (2H), 7.76 (2H), 7.87 (2H), 8.10 (1H), 10.09 (1H), 12.33 (1H).

The starting material for the above title compound is prepared as follows:

### 17a) Ethyl 5-(biphenyl-4-sulfonylamino)-3-phenyl-1H-pyrrolo[2,3-b]pyridine-2-carboxylate

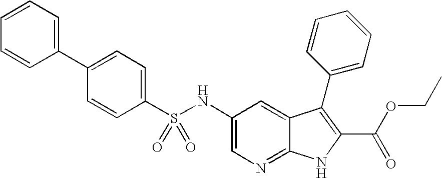

In analogy to example 1f), from 200 mg of the amine prepared in example 4f) and 180 mg of biphenyl-4-sulfonyl chloride, 210 mg of the title compound are obtained.

NMR (300 MHz, DMSO-d6): δ=1.10 (3H), 4.15 (2H), 7.22-7.32 (5H), 7.36 (1H), 7.41 (1H), 7.47 (2H), 7.66 (2H), 7.69 (2H), 7.83 (2H), 8.16 (1H), 10.10 (1H), 12.58 (1H).

### 17b) 5-(Biphenyl-4-sulfonylamino)-3-phenyl-1H-pyrrolo[2,3-b]pyridine-2-carboxylic acid

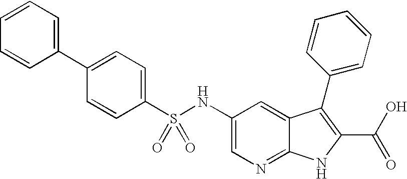

In analogy to example 1j), from 196 mg of the ester prepared in example 17a), 184 mg of the title compound are obtained.

NMR (300 MHz, DMSO-d6): δ=7.27-7.36 (5H), 7.39 (1H), 7.41-7.56 (3H), 7.70 (2H), 7.73 (2H), 7.87 (2H), 8.18 (1H), 10.10 (1H), 12.45 (1H), 13.08 (1H).

### EXAMPLE 18

### (1S,2R)—N-(2-Hydroxycyclopentyl)-5-(biphenyl-4-sulfonylamino)-3-phenyl-1H-pyrrolo[2,3-b]pyridine-2-carboxamide

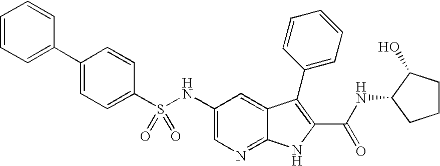

In analogy to example 1), from 50 mg of the acid prepared in example 17b) and 14.7 mg of (1S,2R)-(2-hydroxycyclopentyl)amine, 22 mg of the title compound are obtained.

NMR (300 MHz, DMSO-d6): δ=1.34-1.84 (6H), 3.91 (1H), 3.99 (1H), 4.56 (1H), 7.16 (1H), 7.25-7.38 (4H), 7.39 (1H), 7.42-7.56 (4H), 7.71 (2H), 7.74 (2H), 7.87 (2H), 8.12 (1H), 10.08 (1H), 12.35 (1H).

### EXAMPLE 19

### N-(2-Morpholin-4-ylethyl)-3-phenyl-5-(4-trifluoromethoxyphenyl-sulfonylamino)-1H-pyrrolo[2,3-b]pyridine-2-carboxamide

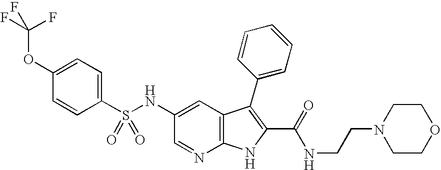

In analogy to example 1), from 50 mg of the acid prepared in example 19b) and 13.6 mg of 2-(morpholin-4-yl)ethylamine, 20 mg of the title compound are obtained.

NMR (300 MHz, DMSO-d6): δ=2.28 (4H), 2.34 (2H), 3.31 (2H), 3.47 (4H), 7.30-7.61 (9H), 7.76 (2H), 8.05 (1H), 10.18 (1H), 12.37 (1H).

The starting material for the above title compound is prepared as follows:

### 19a) Ethyl 3-phenyl-5-(4-trifluoromethoxyphenylsulfonylamino)-1H-pyrrolo-[2,3-b]pyridine-2-carboxylate

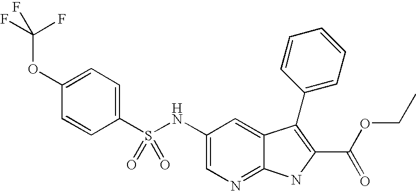

In analogy to example 1f), from 200 mg of the amine prepared in example 4f) and 185 mg of 4-trifluoromethoxyphenylsulfonyl chloride, 232 mg of the title compound are obtained.

NMR (300 MHz, DMSO-d6): δ=1.11 (3H), 4.16 (2H), 7.29 (2H), 7.33-7.42 (4H), 7.52 (2H), 7.71 (2H), 8.11 (1H), 10.18 (1H), 12.60 (1H).

### 19b) 3-Phenyl-5-(4-trifluoromethoxyphenylsulfonylamino)-1H-pyrrolo[2,3-b]-pyridine-2-carboxylic acid

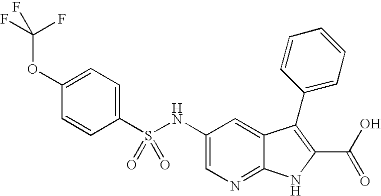

In analogy to example 1j), from 216 mg of the ester prepared in example 19a), 202 mg of the title compound are obtained.

NMR (300 MHz, DMSO-d6): δ=7.28 (2H), 7.31-7.42 (4H), 7.52 (2H), 7.71 (2H), 8.08 (1H), 10.15 (1H), 12.44 (1H), 13.09 (1H).

### EXAMPLE 20

### (1S,2R)—N-(2-Hydroxycyclopentyl)-3-phenyl-5-(4-trifluoromethoxy-phenylsulfonylamino)-1H-pyrrolo[2,3-b]pyridine-2-carboxamide

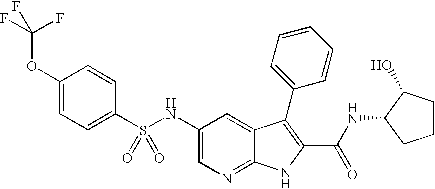

In analogy to example 1), from 70 mg of the acid prepared in example 19b) and 20.2 mg of (1S,2R)-(2-hydroxycyclopentyl)amine, 15 mg of the title compound are obtained.

NMR (300 MHz, DMSO-d6): δ=1.35-1.85 (6H), 3.91 (1H), 4.00 (1H), 4.57 (1H), 7.17 (1H), 7.25-7.48 (6H), 7.57 (2H), 7.75 (2H), 8.06 (1H), 10.16 (1H), 12.38 (1H).

### EXAMPLE 21

### N-(2-Morpholin-4-ylethyl)-3-phenyl-5-(4-trifluoromethylphenyl-sulfonylamino)-1H-pyrrolo[2,3-b]pyridine-2-carboxamide

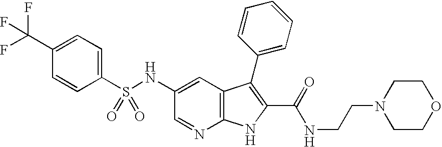

In analogy to example 1), from 50 mg of the acid prepared in example 21b) and 14.1 mg of 2-(morpholin-4-yl)ethylamine, 22 mg of the title compound are obtained.

NMR (300 MHz, DMSO-d6): δ=2.24 (4H), 2.29 (2H), 3.25 (2H), 3.42 (4H), 7.27 (2H), 7.35 (2H), 7.40 (2H), 7.48 (1H), 7.80 (2H), 7.93 (2H), 8.00 (1H), 10.24 (1H), 12.34 (1H).

The starting material for the above title compound is prepared as follows:

### 21a) Ethyl 3-phenyl-5-(4-trifluoromethylphenylsulfonylamino)-1H-pyrrolo[2,3-b]-pyridine-2-carboxylate

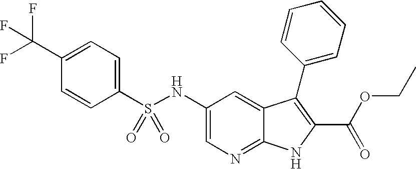

In analogy to example 1f), from 200 mg of the amine prepared in example 4f) and 174 mg of 4-trifluoromethylphenylsulfonyl chloride, 234 mg of the title compound are obtained.

NMR (300 MHz, DMSO-d6): δ=1.11 (3H), 4.16 (2H), 7.26 (2H), 7.31-7.42 (4H), 7.80 (2H), 7.92 (2H), 8.11 (1H), 10.29 (1H), 12.61 (1H).

### 21b) 3-Phenyl-5-(4-trifluoromethylphenylsulfonylamino)-1H-pyrrolo[2,3-b]-pyridine-2-carboxylic acid

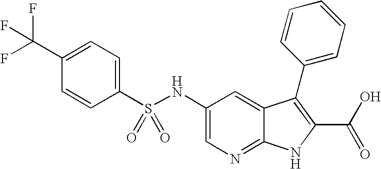

In analogy to example 1j), from 222 mg of the ester prepared in example 21a), 205 mg of the title compound are obtained.

NMR (300 MHz, DMSO-d6): δ=7.26 (2H), 7.28-7.41 (4H), 7.79 (2H), 7.92 (2H), 8.08 (1H), 10.26 (1H), 12.45 (1H), 13.09 (1H).

### EXAMPLE 22

### (1S,2R)—N-(2-Hydroxycyclopentyl)-3-phenyl-5-(4-trifluoromethyl-phenylsulfonylamino)-1H-pyrrolo[2,3-b]pyridine-2-carboxamide

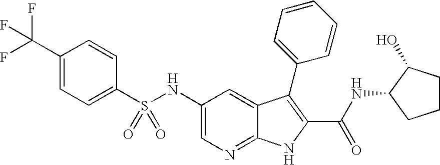

In analogy to example 1), from 60 mg of the acid prepared in example 21b) and 19.9 mg of (1S,2R)-(2-hydroxycyclopentyl)amine, 24 mg of the title compound are obtained.

NMR (300 MHz, DMSO-d6): δ=1.32-1.83 (6H), 3.86 (1H), 3.95 (1H), 4.54 (1H), 7.12 (1H), 7.19-7.44 (6H), 7.79 (2H), 7.92 (2H), 8.02 (1H), 10.23 (1H), 12.36 (1H).

### EXAMPLE 23

### (1S,2R)-5-(Biphenyl-4-sulfonylamino)-3-phenyl-1H-pyrrolo[2,3-b]-pyridine-2-carboxamide

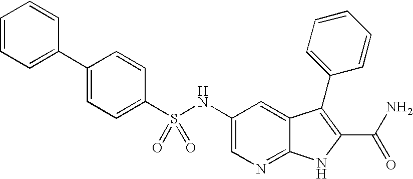

In analogy to example 15), from 63 mg of the acid prepared in example 17b), 23 mg of the title compound are obtained.

NMR (300 MHz, DMSO-d6): δ=7.18 (1H), 7.25-7.38 (5H), 7.39-7.55 (4H), 7.62 (1H), 7.71 (2H), 7.73 (2H), 7.87 (2H), 8.11 (1H), 10.08 (1H), 12.24 (1H).

### BIOLOGICAL EXAMPLES

### Example 1

### sAC Assay

In a suitable buffer system, soluble, sperm-specific adenylate cyclase catalyzes the conversion of adenosine triphosphate (ATP) to cyclic adenosine monophosphate (cAMP) and pyrophosphate. Free cAMP generated in this way is then used in a competitive detection technique, in which the binding of a europium cryptate Eu[K]-labeled anti-cAMP antibody (anti-cAMP-Eu[K]-AB) to a modified allophycocyanine-1 molecule labeled with cAMP molecules (cAMP-XL665) is prevented. In the absence of exogenous cAMP, after excitation at 335 nm there is Fluorescence Resonance Energy Transfer (FRET) between the anti-cAMP-Eu[K]-AB (FRET donor) and the cAMP-XL665 molecule (FRET acceptor). This process is quantified, time-resolved, on the basis of the emission of the FRET acceptor XL665 (665 nm and 620 nm). A decrease in signal (measured as Well Ratio; calculated from the Formula: [(E665 nm/E620 nm)×10000]) can be attributed to the presence of cAMP and thus to the activity of sAC. First, 1.5 μl of the test substance (in 30% DMSO) is placed in each well of a 384-well test plate (polystyrene; 384, NV), and in the solvent controls only 30% DMSO. Then 10 μl of a dilute sAC enzyme solution is applied (enzyme stock solution in 300 mM NaCl, 10% glycerol; pH 7.6; enzyme intermediate and final dilution a) 1:10 and b) 1:2000 in each case in: 1.0 mM MnCl₂; 0.2% BSA; 50 mM Tris pH 7.5 in H₂O). The enzyme reaction is started by adding 5 μl of the ATP substrate solution (200 μM ATP in H₂O) and after incubation (25 min at room temperature) stopped by adding 5 μl of the stop solution (200 μM EDTA in PBS). Finally the whole reaction is adjusted to a total volume of 91.5 μl by adding 70 μl PBS.

Next, 8 μl of detection solution 1 is placed in a well of the 384-well measuring plate (measuring plate: polystyrene; 384, SV—black; detection solution 1: 50 μl cAMP-XL665; 950 μl reconstituted buffer; 2200 μl PBS; cAMP-XL665: prepared by adding 5 ml H₂O to the lyophilized product according to the instructions in Cis bio Kit: #62AMPPEC; storage: in aliquots at −80° C.). Next, 3 μl from the 91.5 μl is added to the corresponding well of the test plate. Finally, 8 μl of detection solution 2 is added (detection solution 2: 50 μl anti-cAMP-Eu[K]-AB; 950 μl reconstituted buffer; 2200 μl PBS; anti-cAMP-Eu[K]-AB: prepared according to the instructions in Cis bio Kit: #62AMPPEC; storage: in aliquots at −80° C.).

After further incubation for 90 min at room temperature, the HTRF result is measured either on the Packard Discovery or with the RubiStar HTRF measuring instrument (delay: 50 μs; integration time: 400 μs).

### Example 2

### Isolation of Human Sperm from Ejaculates and Capacitation

### 2.1. Isolation of the Sperm

Human sperm from ejaculate are purified in a two-layer gradient system based on colloidal silica particles (trade name: Percoll or ISolate).

Per ejaculate, 2.5 ml of pre-warmed lower layer (“90% ISolate lower layer”, from Irvine) is placed in a 15 ml centrifuge tube (conical, plastic) and is carefully covered with 2.5 ml of pre-warmed upper layer (“50% ISolate upper layer”, from Irvine) and held at 37° C. for <1 h on a water bath. The gradient is carefully covered with max. 3 ml of normal (with respect to sperm count, motility and liquefaction) ejaculate. Sedimentation of the sperm is carried out at 1000×g for 25 min at room temperature. Using a glass capillary, the two layers are removed by suction to just above the sperm pellet. For elutriation of the ISolate gradients, the sperm pellets, each resuspended in approx. 200 μl, are transferred to a 15 ml plastic tube with 12 ml mHTF medium (4 mM NaHCO₃; 0.01% BSA; 37° C.) and the sperm are sedimented at 1000×g for 20 min. The medium is removed by suction to just above the pellet and adjusted with mHTF medium (4 mM NaHCO₃; 0.01% BSA; 37° C.) to 1000 μl. The sperm count is determined in a Neubauer counter and for subsequent capacitation is adjusted if necessary to 4×10⁶ sperm/150 μl with mHTF medium (4 mM NaHCO₃; 0.01% BSA; 37° C.).

### 2.2. Capacitation

If the influence of test substances on the acrosome reaction is to be tested, the sperm must be preincubated with the test substances. This preincubation (15 min in a heating cabinet at 37° C.) is necessary to permit penetration of the test substances into the sperm before the start of capacitation, i.e. to achieve presaturation of the binding sites in the sperm, especially in the case of substances that do not pass through the membrane easily. It is also necessary because the increase in BSA concentration during capacitation due to the high lipid binding of the BSA could lead to a decrease in the effective concentration of test substance in the sample.

The test substances are dissolved in DMSO and diluted with mHTF medium (4 mM NaHCO₃; 0.01% BSA; 37° C.), so that in the final 400-μl capacitation sample the DMSO concentration is 0.5%. In each case 150 μl of sperm suspension is added by pipette to 150 μl of the aforementioned temperature-controlled solution of test substance, followed by preincubation at 37° C. for 15 min. Capacitation of the sperm is started by adding 100 μl of mHTF medium (88 mM NaHCO₃; 4% BSA; 37° C.). In the final 400-μl capacitation sample, the concentration of sperm is 10×10⁶/ml, the bicarbonate concentration is 4 mM and the BSA concentration is 1%. Capacitation is carried out for 3 hours at 37° C. in the heating cabinet.

For stopping capacitation, the samples (each of 400 μl) are each transferred completely to a 15-ml sample tube with 1.5 ml mHTF (4 mM NaHCO₃; 37° C.), centrifuged for 5 min at 1000×g and the supernatant is removed. This step removes both the large amount of protein and the test substances.

### Example 3

### Flow Cytometric Determination of the Acrosome Reaction

3.1. Initiation of the Acrosome Reaction by Treatment with Ionophore and Simultaneous CD46-FITC Staining

The acrosome reaction (AR) of the sperm is triggered by binding of the sperm to the zona pellucida (ZP). This releases enzymes from the acrosome, enabling the sperm to penetrate the ZP and reach the ovum. In the AR, there is partial fusion, at the sperm, of the plasma membrane with the outer acrosomal membrane (OAM). At the end the sperm head is still radicalricted by the inner acrosomal membrane (IAM). The CD46 antigen is only detectable at the IAM.

In vitro the acrosome reaction can only be induced with a suitable concentration of the calcium ionophore A23187 on capacitated sperm, but not on uncapacitated sperm or sperm for which capacitation was inhibited by test substances. By means of the FITC-labeled anti-CD46 antibody (from Pharmingen) to the IAM, the acrosome-reacted sperm can be differentiated from the acrosome-intact sperm, in which the IAM is not exposed, in the flow cytometer. With simultaneous staining of the sperm with the DNA stain ethidium homodimer (EhD), which only stains cells that have defective DNA membranes, i.e. are dead, it is possible to distinguish dead sperm from live sperm. Because the ionophore dilutions for initiating the AR appear to be very unstable and must be mixed with the CD46-FITC solution for simultaneous staining, the solutions cannot be prepared before the start of the test, but must be prepared during processing of the capacitation samples.

The sperm pellets are resuspended in the residue of the supernatant and are diluted with 450 μl mHTF (4 mM NaHCO₃; 0.01% BSA; 37° C.) on a water bath (37° C.). 100 μl aliquots of the sperm suspensions are transferred by pipette to prepared sample-FACS flow tubes (on the water bath). 150 μl of a solution with ionophore and FITC-labeled anti-CD46 antibody is added by pipette to the sperm. The final concentration is 800 nm ionophore and a 1:125 dilution of the anti-CD46 antibody in mHTF (4 mM NaHCO₃; 0.01% BSA; 37° C.). The sperm are incubated therein for 30 min, protected from the light, on a water bath at 37° C.

Incubation is stopped by adding 3.5 ml PBS [0.1% BSA]/sample, followed by centrifugation for 5 min at 700×g (room temperature) and then removal of the supernatants with suction. After centrifugation, the samples are kept warm on a hot-plate until measurement.

### 3.2. EhD Staining (for Differentiation of the Dead/Live Acrosome-Reacted Sperm)

500 μl of freshly prepared EhD solution (150 nm EhD in PBS [w/o BSA]; 37° C.) is added to each of the sperm pellets after removal by suction. The samples can then be measured in the flow cytometer (BD FacsCalibur). Measurement is performed at a laser excitation wavelength of 488 nm, detecting 10000 sperm per measurement. Acrosome-reacted sperm are measured via CD46-FITC in the FL-1 filter at 530 nm. Dead sperm are measured by means of EhD-DNA staining in the FL-2 filter at 634 nm. The measurement channels are correspondingly compensated relative to one another beforehand.

### 3.3 Evaluation

The sperm are selected as a very uniform cell population in an FSC-H (forward scatter) versus SSC-H (sideward scatter) dot-blot. As two-color fluorescence staining is used, evaluation is performed by quadrant analysis in an FL-1 (EhD; X axis) vs. FL-2 (FITC-CD46, Y axis) dot-blot with the selected sperm population from the FSC vs. SSC dot-blot:

| | Quadrant in FL-1 | | |
|---|---|---|---|
| | vs. FL-2 dot-blot | Staining | Analysis |
| Q1 = UL | upper left | only EhD | dead, not acrosome-reacted |
| | | | sperm |
| Q2 = UR | upper right | EhD and | dead, acrosome-reacted |
| | | FITC-CD46 | sperm |
| Q3 = LL | lower left | unstained | live, not acrosome-reacted |
| | | | sperm |
| Q4 = LR | lower right | only | live, acrosome-reacted |
| | | FITC-CD46 | sperm |

To calculate the %-induced acrosome-reacted sperm (=“IAR[%]”), only the live sperm from Q3 and Q4 are taken, and their total count is set equal to 100%. IAR is then calculated as follows:

$$I A R [ \% ] = \frac{L R \times 100}{L L + L R}$$

A proportion of the sperm undergo the acrosome reaction spontaneously without addition of ionophore (=“SAR[%]”). Therefore a control measurement is always performed on identically-treated sperm without addition of ionophore. Calculation of SAR is similar to calculation of IAR. The acrosome reaction actually induced by the ionophore (=“ARIC[%]”) is calculated as the difference: ARIC=IAR−SAR.

For subsequent analysis of the influence of our inhibitors on sAC-mediated capacitation (measured as the capacity of the sperm for the ionophore-induced acrosome reaction), the percentage of acrosome-reacted sperm in the positive capacitation control (=incubation with mHTF medium with 25 mM NaHCO₃; 1% BSA without test substances) is set=100%. The capacity of the sperm to which the test substances have been added, for the acrosome reaction, is stated relative to this maximum acrosome reaction.

### Materials Used

mHTF=modif. human tubular fluid (from Irvine Scientific), Dulbeccos's Phosphate-Buffered-Saline (from Gibco) (with Ca²⁺, Mg²⁺, 1 g/L D-glucose, 36 mg/l Na-pyruvate, w/o phenol red, w/o NaHCO₃); bovine serum albumin, Fraction V (from Fluka); dimethyl sulfoxide (DMSO), anhydrous (from Merck); sodium bicarbonate 7.5% solution (893 mM) (from Irvine Scientific); Isolate-Gradient (from Irvine Scientific); Ionophore-A23187 free acid, (from Calbiochem); Ethidium Homodimer (EhD) (from Molecular Probe), Mouse Anti Human CD46:FITC (from Pharmingen).

### REFERENCES

J. W. Carver-Ward, Human Reproduction Vol. 11, No. 9, pp: 1923 ff, 1996 High fertilization prediction by flow cytometric analysis of the CD46 antigen on the inner acrosomal membrane of spermatozoa
O. J. D'Cruz, G. G. Haas, Fertility and Sterility Vol. 65, No. 4, pp: 843 ff, 1996 Fluorescence-labeled fucolectins are superior markers for flow cytometric quantitation of the sperm acrosome reaction
E. Nieschlag, H. M. Behre, Andrology, Springer Verlag 1996

### Examples

| | | IC50 |
|---|---|---|
| Example | Structural formula | (nM) |
| 10 | 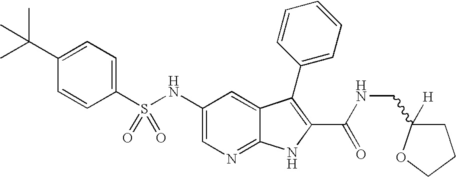 | 64 |
| 22 | 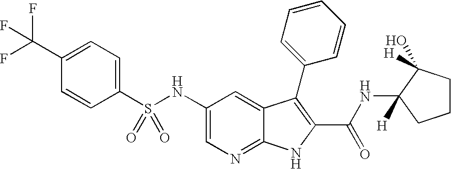 | 25 |
| 8 | 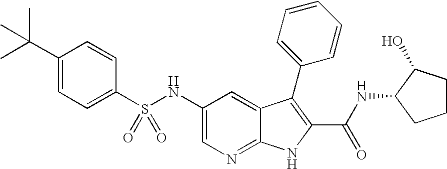 | 13 |
| 6 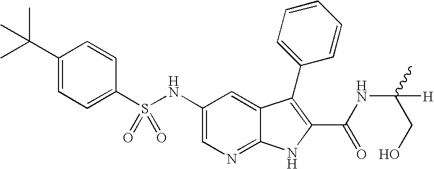 | 37 | |
| Example27fromWO06/032541 | 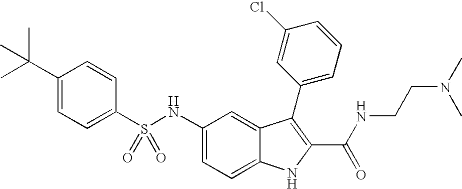 | 630 |
| Example43fromWO06/032541 | 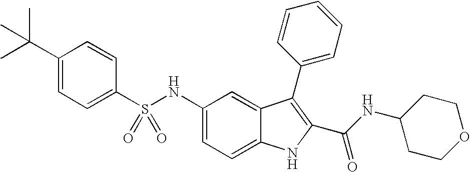 | 86 |

From the table it is apparent that the compounds according to the invention exhibit higher activity, in respect of the inhibition of soluble adenylate cyclase, expressed through the IC₅₀ value, than the existing catechol estrogens (IC50=11 μM) and also than the compounds known from patent application WO 06/032541. The catechol estrogens (OH estradiols) known from the literature are toxic, furthermore, and consequently the compounds according to the invention are superior to the known compounds. The compounds according to the invention are also more potent than the compounds presented by Zippin.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07075764.6, filed Sep. 5, 2007, and U.S. Provisional Application Ser. No. 60/970,005, filed Sep. 5, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula (I), 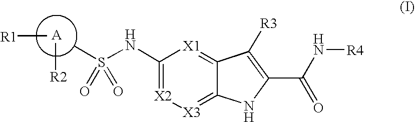
where
A is a C₆-C₁₂-aryl or C₅-C₁₂-heteroaryl radical which can be optionally singly or multiply substituted with R¹ and/or R²,
R¹ is a hydrogen, halogen, amino,
an —S(O)ₚ—C₁-C₆-alkyl group where p is 0-2,
an SO₂NH₂, an —SO₂NH—C₁-C₆-alkyl group, an —NH—SO₂—C₁-C₆-alkyl group, a C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, amino, an —S(O)ₚ—C₁-C₆-alkyl group, where p is 0-2,
an SO₂NH₂, —SO₂NH—C₁-C₆-alkyl group, —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂ or NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH(C₁-C₆-alkyl) or with CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted, or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ is a C₆-C₁₂-aryl,
which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by —O—CH₂—O—, —O—CH₂—CH₂—O—, or —O—C(CH₃)₂—O—, or
a C₅-C₁₆-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or
a C₃-C₆-cycloalkyl, which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
a C₁-C₆-alkyl,
which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl), CO—N(C₁-C₆-alkyl)₂ or C₆-C₁₂-aryl,
which is optionally multiply substituted,
or
C₅-C₁₂-heteroaryl,
which is optionally multiply substituted, or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group.

2. The compounds as claimed in claim 1, where
A is a phenyl, naphthyl or C₅-C₁₂-heteroaryl radical which can be optionally singly or multiply substituted with R¹ and/or R²,
R¹ is a hydrogen, halogen, amino,
an —S(O)ₚ—C₁-C₆-alkyl group, where p is 0-2,
an SO₂NH₂, —SO₂NH—C₁-C₆-alkyl group, —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, amino, an —S(O)ₚ—C₁-C₆-alkyl group, where p is 0-2,
an SO₂NH₂, an —SO₂NH—C₁-C₆-alkyl group, an —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂ or an NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH(C₁-C₆-alkyl) or with CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ is a C₆-C₁₂-aryl,
which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by —O—CH₂—O—, —O—CH₂—CH₂—O—, or —O—C(CH₃)₂—O—,
or
a C₅-C₁₆-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or
a C₃-C₆-cycloalkyl,
which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
a C₁-C₆-alkyl,
which can be optionally singly or multiply, identically or differently substituted with an —S(O)ₚ—C₁-C₆-alkyl group or an —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy,
or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
and also their stereoisomers, diastereomers, enantiomers and salts.

3. The compounds as claimed in claim 1, where
A is a phenyl or a monocyclic C₅-C₇-heteroaryl radical which can be optionally singly or multiply substituted with R¹ and/or R²,
R¹ is a hydrogen, halogen, amino, an —S(O)ₚ—C₁-C₆-alkyl group, where p is 0-2,
an SO₂NH₂, an —SO₂NH—C₁-C₆-alkyl group, an —NH—SO₂—C₁-C₆-alkyl group, C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2,
or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, amino, an —S(O)ₚ—C₁-C₆-alkyl group, where p is 0-2,
an SO₂NH₂, an —SO₂NH—C₁-C₆-alkyl group, an —NH—SO₂—C₁-C₆-alkyl group, C₁-C₆-acyl-, NH—CO—NH₂, —O—CO—NH(C₁-C₆-alkyl), —O—CO—N(C₁-C₆-alkyl)₂ or NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH(C₁-C₆-alkyl) or with CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂,
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R³ is a C₆-C₁₂-aryl,
which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₂-C₆-alkyl)₂ or C₁-C₆-acyl,
which is optionally singly or multiply substituted,
or
with two adjacent positions can be substituted by —O—CH₂—O—, —O—CH₂—CH₂—O—, or —O—C(CH₃)₂—O—, or
a C₅-C₁₆-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂, or
a C₃-C₆-cycloalkyl,
which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
a C₁-C₆-alkyl,
which can be optionally singly or multiply, identically or differently substituted with an —S(O)ₚ—C₁-C₆-alkyl group or an —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
which is optionally multiply substituted,
C₅-C₁₂-heteroaryl,
which is optionally multiply substituted, or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
and also their stereoisomers, diastereomers, enantiomers and salts.

4. The compounds as claimed in claim 1, where
A is a phenyl or a monocyclic C₅-C₇-heteroaryl radical which can be optionally singly or multiply substituted with R¹ and/or R²,
R¹ is a hydrogen, halogen, amino, an —S(O)ₚ—C₁-C₆-alkyl group, where p is 0-2,
an SO₂NH₂, an —SO₂NH—C₁-C₆-alkyl group, an —NH—SO₂—C₁-C₆-alkyl group, C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂, or
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₆-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, C₁-C₆-alkyl, C₁-C₆-alkoxy, C₁-C₆-acyl-, C₆-C₁₂-aryl, C₅-C₁₆-heteroaryl, C₃-C₆-cycloalkyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, O—C₆-C₁₂-aryl, O—C₅-C₁₂-heteroaryl, or
R³ is a C₆-C₁₂-aryl,
which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by
—O—CH₂—O—, —O—CH₂—CH₂—O—, or —O—C(CH₃)₂—O—, or
a C₅-C₁₆-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or
a C₃-C₆-cycloalkyl,
which can be optionally singly or multiply, identically or differently substituted with halogen, CF₃, hydroxy, cyano, CO₂—(C₁-C₆-alkyl), C₁-C₆-alkyl, C₁-C₆-acyl, N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂ or C₁-C₆-alkoxy,
R⁴ is a hydrogen,
a C₁-C₆-alkyl,
which can be optionally singly or multiply, identically or differently substituted with an —S(O)ₚ—C₁-C₆-alkyl group or an —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
and also their stereoisomers, diastereomers, enantiomers and salts.

5. The compounds as claimed in claim 1, where
A is a phenyl or a monocyclic C₅-C₇-heteroaryl radical which can be optionally singly or multiply substituted with R¹ and/or R²,
R¹ is a hydrogen, halogen, amino, an —S(O)ₚ—C₁-C₆-alkyl group, where p is 0-2,
an SO₂NH₂, an —SO₂NH—C₁-C₆-alkyl group, an —NH—SO₂—C₁-C₆-alkyl group, C₁-C₆-acyl, NH—CO—NH₂, —O—CO—NH(C₁-C₆-alkyl), —O—CO—N(C₁-C₆-alkyl)₂, NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with COOH, CO—NH₂, CO—NH—(C₁-C₆-alkyl), CO—N(C₁-C₆-alkyl)₂, C₁-C₆-acyl, C₁-C₆-alkoxy, hydroxy, cyano, CO₂—(C₁-C₆-alkyl), N—(C₁-C₆-alkyl)₂, C₅-C₁₂-heteroaryl,
which is optionally multiply substituted,
C₆-C₁₂-aryl,
which is optionally multiply substituted,
or
a C₁-C₆-alkoxy,
which can be optionally singly or multiply, identically or differently substituted with hydroxy, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or with CO—N(C₁-C₆-alkyl)₂, or
an O—C₆-C₁₂-aryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
an O—C₅-C₁₂-heteroaryl,
which can be optionally singly or multiply, identically or differently substituted with halogen, hydroxy, cyano, COOH, CO—NH₂, C₁-C₆-alkyl, C₃-C₆-cycloalkyl, C₁-C₆-acyl or C₁-C₆-alkoxy, or
a C₆-C₁₂-aryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a C₅-C₁₂-heteroaryl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen, halogen, C₁-C₆-acyl-, NH—CO—NH₂, —O—CO—NH(C₁-C₆-alkyl), —O—CO—N(C₁-C₆-alkyl)₂, an NH—CO—C₁-C₆-alkyl radical, hydroxy, cyano, O—CO—(C₁-C₆-alkyl), CO₂—(C₁-C₆-alkyl), CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, C₁-C₆-alkyl, C₁-C₆-alkoxy, O—C₆-C₁₂-aryl, O—C₅-C₁₂-heteroaryl, C₆-C₁₂-aryl, C₅-C₁₆-heteroaryl, C₃-C₆-cycloalkyl,
R³ is a C₆-C₁₂-aryl,
which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by
—O—CH₂—O—, —O—CH₂—CH₂—O—, or —O—C(CH₃)₂—O—, or
a C₅-C₁₂-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
a C₁-C₆-alkyl,
which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, or C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X¹ is a nitrogen if X² and X³ are a CH group, or
X² is a nitrogen if X¹ and X³ are a CH group, or
X³ is a nitrogen if X¹ and X² are a CH group,
and also their stereoisomers, diastereomers, enantiomers and salts.

6. The compounds as claimed in claim 1, where
A is a phenyl or a monocyclic C₅-C₇-heteroaryl radical which can be optionally singly or multiply substituted with R¹ and/or R²,
R¹ is a hydrogen, halogen, an —S(O)ₚ—C₁-C₆-alkyl group, where p is 0-2,
an SO₂NH₂, an —SO₂NH—C₁-C₆-alkyl group, an —NH—SO₂—C₁-C₆-alkyl group, or
a C₁-C₆-acyl, hydroxy, cyano, CO—NH—(C₁-C₆-alkyl), CO—N(C₁-C₆-alkyl)₂, CO—NH(C₅-C₁₂-heteroaryl), NH—(C₁-C₆-alkyl), N—(C₁-C₆-alkyl)₂, or
a C₁-C₆-alkyl,
which can be optionally singly or multiply, identically or differently substituted with C₁-C₆-alkoxy, hydroxy, cyano, N—(C₁-C₆-alkyl)₂, C₅-C₁₂-heteroaryl,
which is optionally singly or multiply, identically or differently substituted with halogen or C₁-C₆-alkyl,
C₆-C₁₂-aryl,
which is optionally singly or multiply, identically or differently substituted with halogen or C₁-C₆-alkyl, or
a C₁-C₆-alkoxy, or
an O—C₆-C₁₂-aryl,
which can be optionally singly or multiply, identically or differently substituted with halogen or C₁-C₆-alkyl, C₃-C₆-cycloalkyl or C₁-C₆-alkoxy, or
an O—C₅-C₁₂-heteroaryl,
which can be optionally singly or multiply, identically or differently substituted with halogen or C₁-C₆-alkyl, C₃-C₆-cycloalkyl or C₁-C₆-alkoxy, or
a phenyl,
which can be optionally singly or multiply, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a monocyclic C₅-C₇-heteroaryl radical,
which can be optionally singly or multiply, identically or differently substituted with halogen, with C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, with C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen,
R³ is a C₆-C₁₂-aryl,
which can be optionally singly or multiply, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by
—O—CH₂—O—, —O—CH₂—CH₂—O—, or —O—C(CH₃)₂—O—, or
a C₅-C₁₂-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
a C₁-C₆-alkyl,
which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X² is a nitrogen if X¹ and X³ are a CH group, or
X³ is a nitrogen if X¹ and X² are a CH group,
and also their stereoisomers, diastereomers, enantiomers and salts.

7. The compounds as claimed in claim 1, where
A is a phenyl, thienyl or pyridinyl radical which can be optionally singly or doubly substituted with R¹ and/or R²,
R¹ is a hydrogen, halogen, C₁-C₆-alkoxy
a C₁-C₆-alkyl,
which can be optionally singly or multiply, identically or differently substituted with C₁-C₆-alkoxy, hydroxy, cyano, N—(C₁-C₆-alkyl)₂, phenyl,
an O—C₆-C₁₂-aryl or an O—C₅-C₁₂-heteroaryl, or
a phenyl,
which can be optionally singly or doubly, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a thienyl or pyridinyl radical,
which can be optionally singly or doubly, identically or differently substituted with halogen, with C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, with C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen,
R³ is a C₆-C₁₂-aryl,
which can be optionally singly or doubly, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by
—O—CH₂—O—, —O—CH₂—CH₂—O—, or —O—C(CH₃)₂—O—, or
a C₅-C₁₂-heteroaryl,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
a C₁-C₆-alkyl,
which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X² is a nitrogen if X¹ and X³ are a CH group, or
X³ is a nitrogen if X¹ and X² are a CH group, and also their stereoisomers, diastereomers, enantiomers and salts.

8. The compounds as claimed in claim 1, where
A is a phenyl, thienyl or pyridinyl radical which can be optionally singly or doubly substituted with R¹ and/or R²,
R¹ is a hydrogen, halogen, C₁-C₆-alkoxy
a C₁-C₆-alkyl,
which can be optionally singly or multiply, identically or differently substituted with C₁-C₆-alkoxy, hydroxy, cyano, N—(C₁-C₆-alkyl)₂, phenyl,
an O—C₆-C₁₂-aryl or an O—C₅-C₁₂-heteroaryl, or
a phenyl,
which can be optionally singly or doubly, identically or differently substituted with halogen, C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH—(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a thienyl or pyridinyl radical,
which can be optionally singly or doubly, identically or differently substituted with halogen, with C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxy, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, with C₁-C₆-alkyl, hydroxy, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R² is a hydrogen,
R³ is a phenyl,
which can be optionally singly or doubly, identically or differently substituted with halogen, C₁-C₆-alkyl, C₁-C₆-alkoxy, hydroxy, C₁-C₆-alkylene-OH, C₁-C₆-alkylene-NR⁵R⁶, cyano, CO₂—(C₁-C₆-alkyl), N—(C₁-C₆-alkyl)₂, CO—NH—(C₁-C₆-alkyl), CO—NH—(C₁-C₆-alkylene-OH), CO—NH—(C₁-C₆-alkylene-NR⁵R⁶), CO—N(C₁-C₆-alkyl)₂ or C₁-C₆-acyl,
which is optionally singly or multiply substituted,
or
with two adjacent positions can be substituted by
—O—CH₂—O—, —O—CH₂—CH₂—O—, or —O—C(CH₃)₂—O—, or
a thiophenyl, furanyl or pyridinyl radical,
which can be optionally singly or multiply, identically or differently substituted with halogen, C₁-C₆-alkyl, C₁-C₆-acyl, C₁-C₆-alkoxy, hydroxy, cyano, CO₂—(C₁-C₆-alkyl), N—(C₁-C₆-alkyl)₂, CO—NH—(C₁-C₆-alkylene-OH), CO—NH—(C₁-C₆-alkylene-NR⁵R⁶), CO—NH(C₁-C₆-alkyl) or CO—N(C₁-C₆-alkyl)₂,
R⁴ is a hydrogen,
a C₁-C₆-alkyl,
which can be optionally singly or multiply, identically or differently substituted with an —S(O)ₚ—C₁-C₆-alkyl group or —S(O)ₚ—C₃-C₆-cycloalkyl group, where p is 0-2, hydroxy, cyano, COOH, CO—NH₂, SO₂NH₂, SO₂NH—(C₁-C₆-alkyl), C₃-C₆-cycloalkyl, C₁-C₆-acyl, C₁-C₆-alkoxy, CO₂—(C₁-C₆-alkyl), N—(C₁-C₆-alkyl)₂, NH—(C₁-C₆-alkyl), NH—(C₃-C₆-cycloalkyl), CO—NH(C₁-C₆-alkyl) or with CO—N(C₁-C₆-alkyl)₂, C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₆-alkyl, C₁-C₆-acyl or C₁-C₆-alkoxy, or
a C₆-C₁₂-aryl,
which can be optionally singly or multiply, identically or differently substituted with halogen, hydroxy, C₁-C₆-alkyl, C₁-C₆-acyl, C₁-C₆-alkoxy, N—(C₁-C₆-alkyl)₂, NH—(C₁-C₆-alkyl) or cyano, or
a C₅-C₁₂-heteroaryl,
which can be optionally singly or multiply, identically or differently substituted with halogen, amino, hydroxy, cyano, COOH, C₁-C₆-alkyl, C₁-C₆-acyl, C₁-C₆-alkoxy, N—(C₁-C₆-alkyl)₂ or NH—(C₁-C₆-alkyl),
R⁵, R⁶ are independently of one another a hydrogen or a C₁-C₆-alkyl, and
X² is a nitrogen if X¹ and X³ are a CH group, or
X³ is a nitrogen if X¹ and X² are a CH group,
and also their stereoisomers, diastereomers, enantiomers and salts.

9. The compounds as claimed in claim 1, where
A is a phenyl, thienyl or pyridinyl radical which can be optionally singly or doubly substituted with R¹ and/or R²,
R¹ is a hydrogen, halogen, C₁-C₄-alkoxy
a C₁-C₄-alkyl,
which can be optionally singly or multiply, identically or differently substituted with C₁-C₄-alkoxy, hydroxy, cyano, N—(C₁-C₄-alkyl)₂, phenyl,
an O—C₆-C₁₂-aryl or an O—C₅-C₁₂-heteroaryl, or
a phenyl,
which can be optionally singly or doubly, identically or differently substituted with halogen, C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxy, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH—(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl) or an —S(O)ᵣ—CH₃, where r is 0-2, or
two adjacent positions can be substituted by —O—CH₂—O— or —O—C(CH₃)₂—O—, or
a thienyl or pyridinyl radical,
which can be optionally singly or doubly, identically or differently substituted with halogen, with C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxy, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl) or an —S(O)ᵣ—CH₃, where r is 0-2, or
a C₃-C₆-cycloalkyl,
which can be optionally singly or multiply, identically or differently substituted with halogen, with C₁-C₄-alkyl, hydroxy, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R² is a hydrogen,
R³ is a phenyl,
which can be optionally singly or doubly, identically or differently substituted with halogen, C₁-C₄-alkyl, C₁-C₄-alkoxy, hydroxy, C₁-C₄-alkylene-OH, C₁-C₄-alkylene-NR⁵R⁶, cyano, CO₂—(C₁-C₄-alkyl), N—(C₁-C₄-alkyl)₂, CO—NH—(C₁-C₄-alkyl), CO—NH—(C₁-C₄-alkylene-OH), CO—NH—(C₁-C₄-alkylene-NR⁵R⁶), CO—N(C₁-C₄-alkyl)₂ or C₁-C₄-acyl,
which is optionally singly or multiply substituted,
or
with two adjacent positions can be substituted by
—O—CH₂—O—, —O—CH₂—CH₂—O—, or —O—C(CH₃)₂—O—, or
a thiophenyl, furanyl or pyridinyl radical,
which can be optionally singly or multiply, identically or differently substituted with halogen, C₁-C₄-alkyl, C₁-C₄-acyl, C₁-C₄-alkoxy, hydroxy, cyano, CO₂—(C₁-C₄-alkyl), N—(C₁-C₄-alkyl)₂, CO—NH—(C₁-C₄-alkylene-OH), CO—NH—(C₁-C₄-alkylene-NR⁵R⁶), CO—NH(C₁-C₄-alkyl) or CO—N(C₁-C₄-alkyl)₂,
R⁴ is a hydrogen,
a C₁-C₄-alkyl,
which can be optionally singly or multiply, identically or differently substituted with an —S(O)ₚ—C₁-C₄-alkyl group or —S(O)ₚ—C₃-C₆-cycloalkyl group, where p is 0-2, hydroxy, cyano, COOH, CO—NH₂, SO₂NH₂, SO₂NH—(C₁-C₄-alkyl), C₃-C₆-cycloalkyl, C₁-C₄-acyl, C₁-C₄-alkoxy, CO₂—(C₁-C₄-alkyl), N—(C₁-C₄-alkyl)₂, NH—(C₁-C₄-alkyl), NH—(C₃-C₆-cycloalkyl), CO—NH(C₁-C₄-alkyl) or with CO—N(C₁-C₄-alkyl)₂, C₆-C₁₂-aryl,
which is optionally multiply substituted,
C₅-C₁₂-heteroaryl,
which is optionally multiply substituted,
or
a C₃-C₆-cycloalkyl,
which can be optionally singly or multiply, identically or differently substituted with hydroxy, CH₂—OH, cyano, COOH, CO—NH₂, SO₂NH₂, C₁-C₄-alkyl, C₁-C₄-acyl or C₁-C₄-alkoxy, or
a C₆-C₁₂-aryl,
which can be optionally singly or multiply, identically or differently substituted with halogen, hydroxy, C₁-C₄-alkyl, C₁-C₄-acyl, C₁-C₄-alkoxy, N—(C₁-C₄-alkyl)₂, NH—(C₁-C₄-alkyl) or cyano, or
a C₅-C₁₂-heteroaryl,
which can be optionally singly or multiply, identically or differently substituted with halogen, amino, hydroxy, cyano, COOH, C₁-C₄-alkyl, C₁-C₄-acyl, C₁-C₄-alkoxy, N—(C₁-C₄-alkyl)₂ or NH—(C₁-C₄-alkyl),
R⁵, R⁶ are independently of one another a hydrogen or a C₁-C₄-alkyl, and
X² is a nitrogen if X¹ and X³ are a CH group, or
X³ is a nitrogen if X¹ and X² are a CH group,
and also their stereoisomers, diastereomers, enantiomers and salts.

10. The compounds as claimed in claim 1, selected from a group which contains the following compounds:
1. N-(Tetrahydropyran-4-yl)-5-(4-tert-butylphenylsulfonylamino)-3-phenyl-1H-pyrrolo[3,2-b]pyridine-2-carboxamide
2. N-(2-Morpholin-4-ylethyl)-5-(4-tert-butylphenylsulfonylamino)-3-phenyl-1H-pyrrolo[3,2-b]pyridine-2-carboxamide
3. N-(2-Morpholin-4-ylethyl)-5-(4-tert-butylphenylsulfonylamino)-3-phenyl-1H-pyrrolo[2,3-c]pyridine-2-carboxamide
4. N-(2-Morpholin-4-ylethyl)-5-(4-tert-butyl-phenylsulfonylamino)-3-phenyl-1H-pyrrolo[2,3-b]pyridine-2-carboxamide
5. (±)-N-(2-Hydroxypropyl)-5-(4-tert-butylphenylsulfonylamino)-3-phenyl-1H-pyrrolo[2,3-b]pyridine-2-carboxamide
6. N-(Tetrahydropyran-4-yl)-5-(4-tert-butylphenylsulfonylamino)-3-phenyl-1H-pyrrolo[2,3-b]pyridine-2-carboxamide
7. (±)-N-(2-Hydroxy-1-methylethyl)-5-(4-tert-butylphenylsulfonylamino)-3-phenyl-1H-pyrrolo[2,3-b]pyridine-2-carboxamide
8. (1S,2R)—N-(2-Hydroxycyclopentyl)-5-(4-tert-butylphenylsulfonylamino)-3-phenyl-1H-pyrrolo[2,3-b]pyridine-2-carboxamide
9. N-(2-Morpholin-4-ylethyl)-3-phenyl-5-(4-trifluoromethylphenylsulfonylamino)-1H-pyrrolo[2,3-b]pyridine-2-carboxamide
10. (1S,2R)—N-(2-Hydroxycyclopentyl)-5-(biphenyl-4-sulfonylamino)-3-phenyl-1H-pyrrolo[2,3-b]pyridine-2-carboxamide
11. N-(2-Morpholin-4-ylethyl)-3-phenyl-5-(4-trifluoromethoxyphenylsulfonylamino)-1H-pyrrolo[2,3-b]pyridine-2-carboxamide
12. N-(2-Morpholin-4-ylethyl)-5-(biphenyl-4-sulfonylamino)-3-phenyl-1H-pyrrolo[2,3-b]pyridine-2-carboxamide
13. (1S,2R)—N-(2-Hydroxycyclopentyl)-3-phenyl-5-(4-trifluoromethylphenyl-sulfonylamino)-1H-pyrrolo[2,3-b]pyridine-2-carboxamide
14. N-[2-(Morpholine-4-sulfonyl)ethyl]-5-(4-tert-butylphenylsulfonylamino)-3-phenyl-1H-pyrrolo[2,3-b]pyridine-2-carboxamide
15. N-(Pyridin-4-yl)-5-(4-tert-butylphenylsulfonylamino)-3-phenyl-1H-pyrrolo[2,3-b]pyridine-2-carboxamide
16. N-(Thiazol-2-yl)-5-(4-tert-butylphenylsulfonylamino)-3-phenyl-1H-pyrrolo[2,3-b]pyridine-2-carboxamide
17. N-(Pyridin-3-ylmethyl)-5-(4-tert-butyl-phenylsulfonylamino)-3-phenyl-1H-pyrrolo[2,3-b]pyridine-2-carboxamide
18. (±)-N-(Tetrahydrofuran-2-ylmethyl)-5-(4-tert-butylphenylsulfonylamino)-3-phenyl-1H-pyrrolo[2,3-b]pyridine-2-carboxamide
19. N-(Ethyl)-5-(4-tert-butylphenylsulfonylamino)-3-phenyl-1H-pyrrolo[2,3-b]pyridine-2-carboxamide
20. N-[2-(2-Oxoimidazolidin-1-yl)ethyl]-5-(4-tert-butylphenylsulfonylamino)-3-phenyl-1H-pyrrolo[2,3-b]pyridine-2-carboxamide
21. (1S,2R)—N-(2-Hydroxycyclopentyl)-3-phenyl-5-(4-trifluoromethoxyphenyl-sulfonylamino)-1H-pyrrolo[2,3-b]pyridine-2-carboxamide
22. 5-(4-tert-Butylphenylsulfonylamino)-3-phenyl-1H-pyrrolo[2,3-b]pyridine-2-carboxamide
23. N-(2-Sulfamoylethyl)-5-(4-tert-butylphenylsulfonylamino)-3-phenyl-1H-pyrrolo[2,3-b]pyridine-2-carboxamide
24. 5-(Biphenyl-4-sulfonylamino)-3-phenyl-1H-pyrrolo[2,3-b]pyridine-2-carboxamide
25. (1S,2R)—N-(2-Hydroxycyclopentyl)-3-benzo[1,3]dioxol-5-yl-5-(4-trifluoro-methoxyphenylsulfonylamino)-1H-pyrrolo[2,3-b]pyridine-2-carboxamide
26. (1S,2R)—N-(2-Hydroxycyclopentyl)-3-thiophen-2-yl-5-(4-trifluoromethoxy-phenylsulfonylamino)-1H-pyrrolo[2,3-b]pyridine-2-carboxamide
27. (1S,2R)—N-(2-Hydroxycyclopentyl)-3-thiophen-3-yl-5-(4-trifluoromethoxy-phenylsulfonylamino)-1H-pyrrolo[2,3-b]pyridine-2-carboxamide
28. (1S,2R)—N-(2-Hydroxycyclopentyl)-3-furan-2-yl-5-(4-trifluoromethoxy-phenylsulfonylamino)-1H-pyrrolo[2,3-b]pyridine-2-carboxamide
29. (1S,2R)—N-(2-Hydroxycyclopentyl)-3-furan-3-yl-5-(4-trifluoromethoxy-phenylsulfonylamino)-1H-pyrrolo[2,3-b]pyridine-2-carboxamide
30. (1S,2R)—N-(2-Hydroxycyclopentyl)-3-benzo[1,3]dioxol-5-yl-5-(4-tert-butyl-phenylsulfonylamino)-1H-pyrrolo[2,3-b]pyridine-2-carboxamide
31. (1S,2R)—N-(2-Hydroxycyclopentyl)-3-thiophen-2-yl-5-(4-tert-butylphenyl-sulfonylamino)-1H-pyrrolo[2,3-b]pyridine-2-carboxamide
32. (1S,2R)—N-(2-Hydroxycyclopentyl)-3-thiophen-3-yl-5-(4-tert-butylphenyl-sulfonylamino)-1H-pyrrolo[2,3-b]pyridine-2-carboxamide
33. (1S,2R)—N-(2-Hydroxycyclopentyl)-3-furan-2-yl-5-(4-tert-butylphenyl-sulfonylamino)-1H-pyrrolo[2,3-b]pyridine-2-carboxamide
34. (1S,2R)—N-(2-Hydroxycyclopentyl)-3-furan-3-yl-5-(4-tert-butylphenyl-sulfonylamino)-1H-pyrrolo[2,3-b]pyridine-2-carboxamide
35. (±)-N-(Tetrahydrofuran-2-ylmethyl)-3-thiophen-3-yl-5-(4-trifluoromethoxy-phenylsulfonylamino)-1H-pyrrolo[2,3-b]pyridine-2-carboxamide
36. (±)-N-(Tetrahydrofuran-2-ylmethyl)-3-thiophen-2-yl-5-(4-trifluoromethoxy-phenylsulfonylamino)-1H-pyrrolo[2,3-b]pyridine-2-carboxamide
37. (±)-N-(Tetrahydrofuran-2-ylmethyl)-3-thiophen-3-yl-5-(4-tert-butylphenyl-sulfonylamino)-1H-pyrrolo[2,3-b]pyridine-2-carboxamide
38. (±)-N-(Tetrahydrofuran-2-ylmethyl)-3-thiophen-2-yl-5-(4-tert-butyl-phenylsulfonylamino)-1H-pyrrolo[2,3-b]pyridine-2-carboxamide

11. The compounds of the general formula I as claimed in claim 1 as medicinal products.

12. The medicinal products as claimed in claim 11, which comprise the compound of general formula 1 at an effective dose.

13. The medicinal products as claimed in claim 11 for reducing or inhibiting the activity of soluble adenylate cyclase.

14. The medicinal products as claimed in claim 11 for the treatment of diseases.

15. The medicinal products as claimed in claim 11, in which the diseases are caused by disturbances in cAMP metabolism.

16. The use of the compounds of general formula I according to claim 1 in suitable vehicles and excipients for contraception.

17. The medicinal products as claimed in claim 11 with suitable vehicles and excipients.

18. The use of the compounds of general formula I as claimed claim 1 for the production of a pharmaceutical preparation for enteral, parenteral, vaginal and oral application.

19. A contraceptive suppository comprising the compounds as claimed in claim 1.

20. The suppository as claimed in claim 19 for female fertility control.

21. A process for the preparation of the compounds of general formula (I) which comprises reacting a compound of the formula II 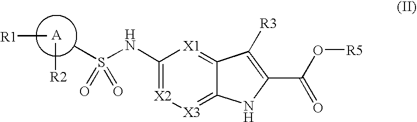
in which R¹, R², R³, X¹, X² and X³ have the definitions given above and R⁵ can be a hydrogen or a C₁-C₆-alkyl radical, preference being given to a hydrogen or to a methyl or ethyl radical, with an amine of the general formula III 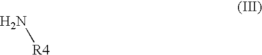
in which R⁴ has the definition given above, and/or any required protecting groups are subsequently cleaved and/or any double bonds present are hydrogenated, and reaction is carried out to give the compounds of general formula (I).

22. Intermediates of general formula (II) 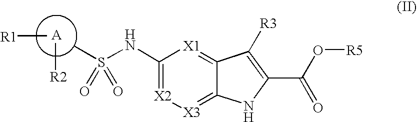
in which R¹, R², R³, X¹, X² and X³ have the definitions given above and R⁵ can be a hydrogen or a C₁-C₆-alkyl radical, preference being given to hydrogen or to the methyl or ethyl radical.

23. The use of the intermediate compounds of general formula (II) according to claim 22 for the preparation of the compounds of general formula (I).

24. A method for controlling fertility of a male patient comprising administering a compound according to claim 1 as an inhibitor of adenylate cyclase.
